SEQUENCE LISTING (1 sequence)

SEQ ID NO: 1 - protein, 267 aa, synthetic construct
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

(19) United States
(12) Patent Application Publication
(10) Pub. No.: US 2026/0250305 A1
(43) Pub. Date: Aug. 27, 2026

(54) COMPOUNDS AND PROCESSES FOR THE SYNTHESIS OF SPHINGOMYELINS

(71) Applicant: ABIONYX PHARMA SA, Balma (FR)

(72) Inventor: Vikas GAJARE, Hyderabad (IN)

(73) Assignee: ABIONYX PHARMA SA, Balma (FR)

(21) Appl. No.: 18/878,230

(22) PCT Filed: Jun. 27, 2023
(86) PCT No.: PCT/IB2023/000381

§ 371 Date: Dec. 23, 2024

Related U.S. Application Data
(60) Provisional application No. 63/356,178, filed on Jun. 28, 2022.

(51) Int. Cl. C07F 9/10; A61K 31/661

(52) U.S. Cl. CPC C07F 9/10; A61K 31/661

(57) ABSTRACT

Novel compounds useful in the synthesis of sphingomyelins, for example N-palmitoyl-D-erythro-sphingomyelin, novel processes for making sphingomyelins using the novel compounds, preparations of sphingomyelin made by the processes, and compositions comprising sphingomyelins.

### 1. CROSS-REFERENCE TO RELATED APPLICATIONS

This application claims the priority benefit of U.S. application No. 63/356,178, filed Jun. 28, 2022, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Jun. 20, 2023 is named CRN-047WO_SL.xml and is 2,162 bytes in size.

### 3. BACKGROUND

Sphingomyelins (SMs) are major phospholipid components of biological membranes and plasma lipoprotein complexes, such as high-density lipoprotein (HDL). Sphingomyelin is also a component of some HDL-mimetic complexes, for example CER-001, an HDL-mimetic comprising apolipoprotein A-I, sphingomyelin, and the negatively charged lipid DPPG.

Commercially available preparations of sphingomyelins are typically mixtures of naturally occurring sphingomyelins, for example from chicken egg, porcine brain, or bovine milk. The actual composition can vary depending on source and can contain various fatty acid chain lengths. N-palmitoyl sphingomyelin is a major component of natural sphingomyelins, with N-palmitoyl-D-erythro-sphingomyelin the main naturally-occurring isomer.

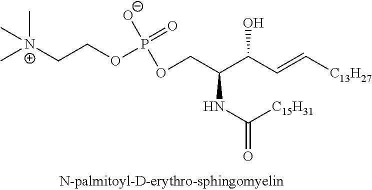

While processes for producing sphingomyelins synthetically have been described (see WO 2014/140787), commercially available sphingomyelins typically remain naturally sourced. Thus, there remains a need for new processes for making sphingomyelins synthetically.

### 4. SUMMARY

The present disclosure provides novel compounds useful in the synthesis of sphingomyelins, for example N-palmitoyl-D-erythro-sphingomyelin, novel processes for making sphingomyelins using the novel compounds, preparations of sphingomyelin made by processes of the disclosure, and compositions comprising sphingomyelins made by processes of the disclosure.

In one aspect, the disclosure provides compounds of Formula (I):

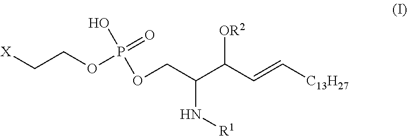

and salts thereof, wherein:
X is halogen, for example Br, CI, F, or I;
R¹ is an acyl group having 3 to 36 carbon atoms and zero to six carbon-carbon double bonds, for example palmitoyl, myristoyl, stearoyl, oleoyl, linoleoyl, linolenoyl, arachidonoyl, or eicosapentaenoyl; and
R² is a protecting group, for example benzoyl.

In further aspects, the disclosure provides processes for synthesizing compounds of Formula (I) and salts thereof.

In some embodiments, compounds of Formula (I) and salts thereof can be synthesized by reacting a compound of Formula (0a):

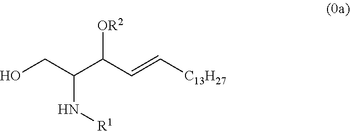

or a salt thereof with

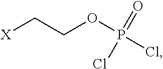

where R¹, R², and X are as defined for compounds of Formula (I).

In other embodiments, compounds of Formula (I) and salts thereof can be synthesized by reacting a compound of Formula (0c):

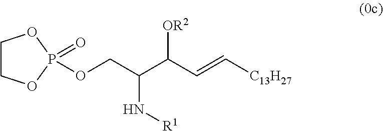

or a salt thereof with MgX₂, where R¹, R², and X are as defined for compounds of Formula (I).

Further exemplary features of compounds of Formula (I) and salts thereof, and processes for synthesizing compounds of Formula (I) and salts thereof are described in Sections 6.1.2 and 6.1.5., and specific embodiments 1 to 7 and 50 to 66, infra.

Compounds of Formula (I) can be used, for example, in the synthesis of sphingomyelins and sphingomyelin precursors, for example protected sphingomyelins.

Accordingly, in further aspects, the disclosure provides processes for synthesizing a sphingomyelin or a salt thereof. The processes typically comprise reacting a compound of Formula (I) or a salt thereof with trimethylamine to produce a compound of Formula (II)

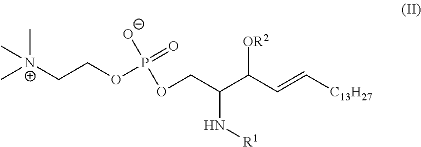

or a salt thereof, where R¹ and R² are as defined for compounds of Formula (I);
   and
removing the R² protecting group from the compound of Formula (II) or a salt thereof to produce a sphingomyelin of Formula (III):

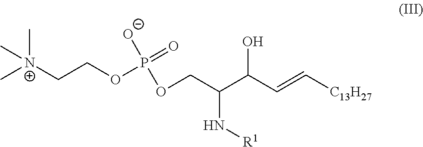

or a salt thereof, wherein R¹ is as defined for compounds of Formula (I).

In further aspects, the disclosure provides processes for synthesizing a protected sphingomyelin or a salt thereof. The processes typically comprise reacting a compound of Formula (I) or a salt thereof with trimethylamine to produce a compound of Formula (II)

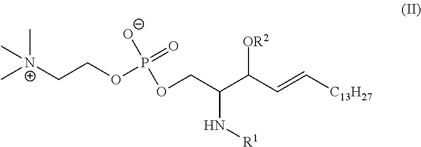

or a salt thereof, wherein R¹ and R² are as defined for compounds of Formula (I).

In further aspects, the disclosure provides sphingomyelins and salts thereof and protected sphingomyelins and salts thereof produced by the processes of the disclosure.

Exemplary features of processes for making sphingomyelins, processes for making protected sphingomyelins and products of such processes are described in Sections 6.1.3 and 6.1.5 and specific embodiments 8 to 30 and 33 to 49, infra.

In further aspects, the disclosure provides compositions comprising a sphingomyelin or salt thereof and processes for their production. For example, the sphingomyelin or salt thereof can be incorporated into a lipid binding-protein based complex, for example CER-001. Exemplary features of such compositions and processes are described in Section 6.1.4 and specific embodiments 31, 32, and 66 to 84, infra.

### 5. BRIEF DESCRIPTION OF THE FIGURES

FIGS. 1A-1B are 1H-NMR spectra of egg sphingomyelin (egg Sph) (FIG. 1A) and synthetic sphingomyelin (sSph) made according to the processes of Example 1 (FIG. 1B).

FIG. 2 shows HPTLC analyses of egg sphingomyelin and synthetic sphingomyelin.

FIGS. 3A-3D show plasma phospholipid (FIG. 3A), human ApoA-I (FIG. 3B), total cholesterol (FIG. 3C) and HDL-total cholesterol (FIG. 3D) levels in rabbits administered CER-001 made with egg sphingomyelin 001 and synthetic sphingomyelin. CER-001 made with egg sphingomyelin (circles and triangles) and CER-001 made with synthetic sphingomyelin (diamonds and open circles) were infused into fasted rabbits at doses of 5 mg/kg (diamonds and circles) or 20 mg/kg (triangles and open circles) and compared to a vehicle group (squares). There were 4 animals per group.

FIGS. 4A-4B show GPC profiles of CER-001 made with from eggSph (FIG. 4A) or sSph (FIG. 4B).

### 6. DETAILED DESCRIPTION

### 6.1.1. Terms

A fatty acid is a carboxylic acid having a long aliphatic tail that can be either saturated or unsaturated. Unsaturated fatty acids have one or more carbon-carbon double bonds, and each carbon-carbon double bond can occur in a cis or trans configuration. A fatty acid residue is a fatty acid less the —OH group of the fatty acid's carboxyl group. As used herein, the term “Ac” or “acyl” refers to a fatty acid residue.

In certain embodiments of the disclosure, the fatty acid or fatty acid residue has 3 to 36 carbons and zero to six carbon-carbon double bonds. Such fatty acid residues can be represented as a radical of general formula-C(O)R, where R is an alkyl group having 2 to 35 carbons and zero to six carbon-carbon double bonds. In particular embodiments of the disclosure, the fatty acid or fatty acid residue has 4 to 28 carbons and zero to six carbon-carbon double bonds. In further embodiments, the fatty acid or fatty acid residue has 11 to 25 carbons and zero to six carbon-carbon double bonds. In still further embodiments, the fatty acid or fatty acid residue has 11 to 25 carbons and one or two carbon-carbon double bonds. In further embodiments, the fatty acid or fatty acid residue has 14 to 20 carbons and zero to six carbon-carbon double bonds. In yet further embodiments, the fatty acid or fatty acid residue has 15 to 17 carbons and zero to six carbon-carbon double bonds. In a particular embodiment, the fatty acid is palmitic acid and the fatty acid residue is palmitoyl.

Suitable fatty acids also include, but are not limited to, omega fatty acids such as ω-3, or ω-6, or ω-9 fatty acids; and essential fatty acids, such as, but not limited to, linoleic acid (LA), a-linolenic acid (ALA), an n-3 fatty acid, e.g., eicosapentaenoic acid (EPA) and docosahexaenoic acid (DHA).

Suitable fatty acids useful in the present disclosure include, but are not limited to, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, myristoleic acid, palmitoleic acid, sapienic acid, oleic acid, elaidic acid, vaccenic acid, linoleic acid, linoelaidic acid, a-linolenic acid, and erucic acid.

If the fatty acid is a monounsaturated fatty acid, it can be a cis- or trans monounsaturated fatty acid such as, but not limited to, oleic acid, elaidic acid, myristoleic acid, palmitoleic acid, sapienic acid, oleic acid, elaidic acid, vaccenic acid, and erucic acid.

The “benzoyl” or “Bz” protecting group refers to a group having the following structure:

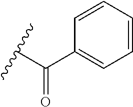 [SMILES: *C(=O)c1ccccc1]

Benzoylating reagents include, but are not limited to, reagents such as benzoyl halides such as benzoyl chloride.

The “Boc” protecting group refers to a tert-butyloxycarbonyl protecting group.

As used herein, “C₁₃H₂₇—” and “C₁₅H₃₁—” mean CH₃(CH₂)₁₂— and CH₃(CH₂)₁₄, respectively.

As used herein, the term “completion” when referring to a reaction, means that the reaction system has either attained equilibrium such that the concentrations of reactants and products remain constant or at least one of the reactants in the reaction has been reduced to less than 20% (by mass) of it's initial amount. In some embodiments, a reaction that has proceeded to completion has less than 20%, less than 10%, less than 5%, less than 3%, less than 2%, or less than 1% of a reactant remaining. In some embodiments of the processes described herein, one or more reaction steps (e.g., all) of the processes reactions described herein are allowed to proceed to completion. Reactions can be monitored, for example, by thin layer chromatography or HPLC, to determine when a reaction is approaching or has reached completion.

Certain compounds of the disclosure can be in the form of a salt. In some embodiments, the salt is a pharmaceutically acceptable salt. Pharmaceutically acceptable salts include, for example, acid-addition salts and base-addition salts. The acid that forms an acid addition salt can be an organic acid or an inorganic acid. A base that forms a base-addition salt can be an organic base or an inorganic base. In some embodiments, a pharmaceutically acceptable salt is a metal salt. In some embodiments, a pharmaceutically acceptable salt is an ammonium salt.

Acid-addition salts can arise from the addition of an acid to the free-base form of a compound. In some embodiments, the acid is organic. In some embodiments, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Non-limiting examples of suitable acid-addition salts include a hydrochloride salt, a hydrobromide salt, a hydroiodide salt, a nitrate salt, a nitrite salt, a sulfate salt, a sulfite salt, a phosphate salt, a hydrogen phosphate salt, a dihydrogen phosphate salt, a carbonate salt, a bicarbonate salt, a nicotinate salt, an isonicotinate salt, a lactate salt, a salicylate salt, a 4-amino salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

Metal salts can arise from the addition of an inorganic base to a compound of the invention having a carboxyl group. The inorganic base consists of a metal cation paired with a basic couterion, such as, for example, hydroxide, carbonate, bicarbonate, or phosphate. The metal can be an alkali metal, alkaline earth metal, transition metal, or main group metal. Non-limiting examples of suitable metals include lithium, sodium, potassium, cesium, cerium, magnesium, manganese, iron, calcium, strontium, cobalt, titanium, aluminum, copper, cadmium, and zinc.

Non-limiting examples of suitable metal salts include a lithium salt, a sodium salt, a potassium salt, a cesium salt, a cerium salt, a magnesium salt, a manganese salt, an iron salt, a calcium salt, a strontium salt, a cobalt salt, a titanium salt, a aluminum salt, a copper salt, a cadmium salt, and a zinc salt.

Ammonium salts can arise from the addition of ammonia or an organic amine to a compound of the invention having a carboxyl group. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include is a triethylammonium salt, a diisopropylammonium salt, an ethanolammonium salt, a diethanolammonium salt, a triethanolammonium salt, a morpholinium salt, an N-methylmorpholinium salt, a piperidinium salt, an N-methylpiperidinium salt, an N-ethylpiperidinium salt, a dibenzylammonium salt, a piperazinium salt, a pyridinium salt, a pyrrazolium salt, an imidazolium salt, a pyrazinium salt, an ethylenediammonium salt, an N,N′-dibenzylethylenediammonium salt, a procaine salt, a chloroprocaine salt, a choline salt, a dicyclohexylammonium salt, and a N-methylgluc amine salt.

Unless required otherwise by context, it should be understood that a disclosure relating to a compound, for example, of Formula (I), of Formula (II), or a sphingomyelin, is also a disclosure relating to salts of the compound.

### 6.1.2. Compounds of Formula (I) and Processes for Making Compounds of Formula (I)

In some aspects, the disclosure provides compounds of Formula (I):

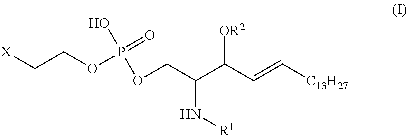

and salts thereof, wherein:
X is halogen, for example Br, CI, F, or I;
R¹ is an acyl group having 3 to 36 carbon atoms and zero to six carbon-carbon double bonds, for example palmitoyl, myristoyl, stearoyl, oleoyl, linoleoyl, linolenoyl, arachidonoyl, or eicosapentaenoyl; and
R² is a protecting group, for example benzoyl.

In preferred embodiments, X is Br.

In preferred embodiments, R¹ is palmitoyl.

In some embodiments, a compound of Formula (I) is a compound of Formula (Ia):

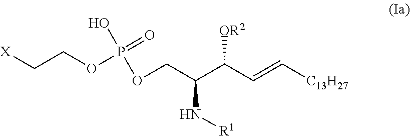

or a salt thereof.

A particularly preferred compound of Formula (I) is

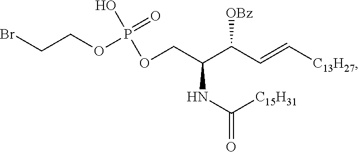

or a salt thereof.

In certain embodiments, a compound of Formula (I) has an enantiomeric purity of at least 85% and contains no more than 15% of its corresponding opposite enantiomer. In further embodiments, a compound of Formula (I) has an enantiomeric purity of at least 90% and contains no more than 10% of its corresponding opposite enantiomer. In yet further embodiments, a compound of Formula (I) has an enantiomeric purity of at least 95% and contains no more than 5% of its corresponding opposite enantiomer. In yet further embodiments, a compound of Formula (I) has an enantiomeric purity of at least 98% and contains no more than 2% of its corresponding opposite enantiomer. Methods for determining enantiomeric purity are known in the art, for example as described in WO 2014/140787.

Compounds of Formula (I) and salts thereof can be synthesized by reacting a compound of Formula (0a):

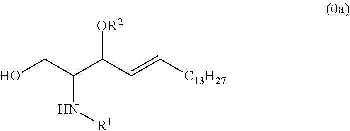

or a salt thereof with

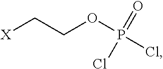

where R¹, R², and X are as defined for compounds of Formula (I).

The process can comprise reacting a compound of Formula (0b)

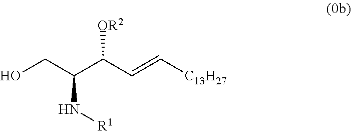

or a salt thereof with

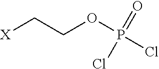

to provide a compound of Formula (I) having the same stereochemistry as the compound of Formula (0b).

Compounds of Formula (I) and salts thereof can also be synthesized by reacting a compound of Formula (0c):

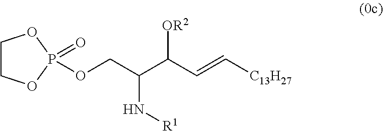

or a salt thereof with MgX₂, where R¹, R², and X are as defined for compounds of Formula (I). In some embodiments, MgX₂ is the MgBr₂, for example in the form of magnesium bromide ethyl etherate. The molar ratio of the compound of Formula (0c) to MgX₂ in some embodiments is 1:1 to 1:3, 2:1 to 1:2, for example 1:1, 1:1.5, or 1:2. The reaction can be carried out at any suitable temperature, for example 20° C. to 40° C. or 25 to 35° C.

Compounds of Formula (0c) can be synthesized, for example, by reacting

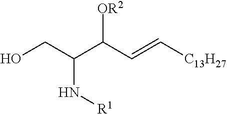

with 2-chloro-2-oxo-1,3,2-dioxaphospholane (CCP), for example in the presence of a base such as trimethylamine (NMe₃) and a solvent such as THF.

The process can comprise reacting a compound of Formula (0d):

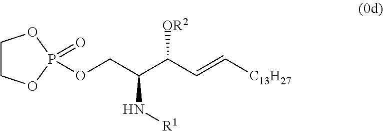

or a salt thereof with MgX₂ to provide a compound of Formula (I) having the same stereochemistry as the compound of Formula (0d).

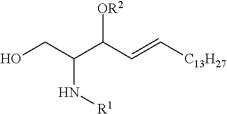

The molar ratio of to CCP can be, for example, in a molar ratio range of 3:1 to 1:3 or 2:1 to 1:2, for example 1:1 or 1:1.5. The reaction can be performed at a suitable temperature, for example at a temperature ranging from −10° C. to 20° C. or 0 to 10° C. The reaction can be monitored by TLC.

Exemplary schemes for synthesizing compounds of Formula (I) and Formula (I) precursors such as compounds of Formula (0) are described in Section 6.1.5.

### 6.1.3. Processes for Synthesizing Sphingomyelins, Protected Sphingomyelins and Products Thereof

The disclosure provides processes for making sphingomyelins using compounds of Formula (I) and salts thereof. The processes typically comprise reacting a compound of Formula (I) or a salt thereof with trimethylamine to produce a compound of Formula (II)

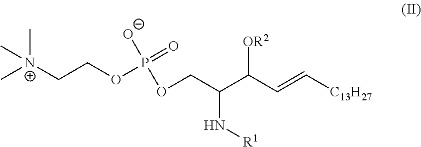

or a salt thereof, where R¹ and R² are as defined for compounds of Formula (I);
   and
removing the R² protecting group from the compound of Formula (II) or a salt thereof to produce a sphingomyelin of Formula (III):

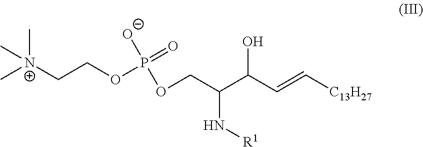

or a salt thereof, wherein R¹ is as defined for compounds of Formula (I).

In some embodiments, step (a) comprises reacting a compound of Formula (Ia)

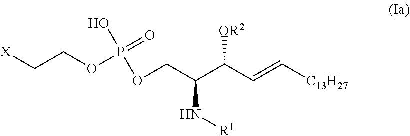

or a salt thereof with trimethylamine to produce a compound of Formula (IIa)

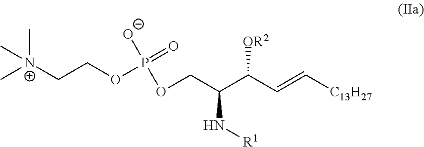

or a salt thereof.

The step of reacting the compound of Formula (I) or a salt thereof with trimethylamine can be performed in a suitable solvent, for example water, methanol, or solvent mixtures such as methanol:THF mixtures (e.g., in a 2:1 vol:vol ratio). Preferably, an excess of trimethylamine is used, e.g., 10 to 100 equivalents, 20 to 80 equivalents, 30 to 70 equivalents, or 40 to 60 equivalents of trimethylamine per equivalent of compound of Formula (I) or salt thereof. In some embodiments, 50 equivalents of trimethylamine are used. The reaction can be performed at any suitable temperature, for example 30° C. to 60° C. or 40° C. to 50° C. TLC can be used to monitor the reaction progress, for example by monitoring disappearance of starting material to determine when the reaction has reached completion. In some embodiments, the reaction can be allowed to proceed to 18 to 30 hours, optionally 22 to 26 hours.

The step of reacting the compound of Formula (I) or a salt thereof with trimethylamine provides a protected sphingomyelin. The protected sphingomyelin can be worked up from the reaction mixture and optionally purified, for example by chromatography (e.g., as described in Sections 6.1.5 and 7).

Removal of the R² protecting group provides sphingomyelin. Removal of a R² benzoyl group can proceed as follows: the benzoyl-protected sphingomyelin can be dissolved in a protic polar solvent and a base is added. The reaction proceeds for 8 to 24 hours at about 22° C. In one embodiment the protic polar solvent is methanol, ethanol, n-propanol, isopropanol, or mixtures thereof. In yet another embodiment the base is sodium methoxide, potassium carbonate, lithium hydroxide. In a particular embodiment, the base is sodium methoxide.

Sphingomyelins produced by the processes described herein, for example N-acyl-D-erythro-sphingomyelins such as N-palmitoyl-D-erythro-sphingomyelin can be worked up and/or purified by one or more means, for example recrystallization, silica gel chromatography, high performance liquid chromatography or other methods known to those skilled in the art. In some embodiments, the workup and/or purification comprises distillation, filtration, washing, and drying, for example as described in Section 7. In some embodiments, the sphingomyelin is washed with acetone, dried under vacuum (e.g., for 30-50 hours at 30° C.), and packaged in a container purged with nitrogen.

The processes of the disclosure can be performed on a commercial scale, for example, to produce commercial scale quantities of an N-acyl-D-erythro-sphingomyelin, in particular, N-palmitoyl-D-erythro-sphingomyelin. For example, the processes can be performed on a scale to provide 1 kg to 50 kg or more of an N-acyl-D-erythro-sphingomyelin, in particular, N-palmitoyl-D-erythro-sphingomyelin, e.g., 1 kg to 25 kg, 1 kg to 10 kg, 5 kg to 25 kg, 5 kg to 10 kg, 10 kg to 25 kg, or 25 kg to 50 kg.

In certain embodiments, the N-palmitoyl-D-erythro-sphingomyelin has an enantiomeric purity of at least 85% and contains no more than 15% of its corresponding opposite enantiomer. In further embodiments, the N-palmitoyl-D-erythro-sphingomyelin has an enantiomeric purity of at least 90% and contains no more than 10% of its corresponding opposite enantiomer. In yet further embodiments, the N-palmitoyl-D-erythro-sphingomyelin has an enantiomeric purity of at least 95% and contains no more than 5% of its corresponding opposite enantiomer. In yet further embodiments, the N-palmitoyl-D-erythro-sphingomyelin has an enantiomeric purity of at least 98% and contains no more than 2% of its corresponding opposite enantiomer. Methods for determining enantiomeric purity are known in the art, for example as described in WO 2014/140787.

### 6.1.4. Compositions Comprising Sphingomyelin

In various aspects, the disclosure provides sphingomyelins produced by the processes of the disclosure and compositions comprising sphingomyelin produced by the processes of the disclosure.

The sphingomyelin can in some embodiments be in crystalline form. Without being bound by theory, it is believed that sphingomyelin in crystalline form indicates that the sphingomyelin is very pure.

The synthetic sphingomyelins produced by the processes of the disclosure can be used in the manufacture of lipid binding protein-based complexes such as CER-001, Apomers, and Cargomers.

CER-001 is described in Example 4 of WO 2012/109162. WO 2012/109162 refers to CER-001 as a complex having a 1:2.7 lipoprotein weight:total phospholipid weight ratio with a SM:DPPG weight:weight ratio of 97:3. Example 4 of WO 2012/109162 also describes a method of manufacturing CER-001. When used in the context of the disclosure, CER-001 refers to a lipoprotein complex whose individual constituents can vary from CER-001 as described in Example 4 of WO 2012/109162 by up to 20%. In certain embodiments, the constituents of the lipoprotein complex vary from CER-001 as described in Example 4 of WO 2012/109162 by up to 10%. Preferably, the constituents of the lipoprotein complex are those described in Example 4 of WO 2012/109162 (plus/minus acceptable manufacturing tolerance variations). The lipoprotein in CER-001, apolipoprotein A-I (ApoA-I), preferably has an amino acid sequence corresponding to amino acids 25 to 267 of SEQ ID NO:1 of WO 2012/109162. SEQ ID NO:1 of WO 2012/109162 is set forth as SEQ ID NO:1 of the present application. ApoA-I can be purified by animal sources (and in particular from human sources) or produced recombinantly. In preferred embodiments, the ApoA-I in CER-001 is recombinant ApoA-I. CER-001 is preferably highly homogeneous, for example at least 80%, at least 85%, at least 90%, at least 95%, at least 97%, at least 98%, or at least 99% homogeneous, as reflected by a single peak in gel permeation chromatography. See, e.g., Section 6.4 of WO 2012/109162.

In particular embodiments, the ApoA-I in CER-001 is recombinant ApoA-I produced by a mammalian host cell. The host cell can be from any mammalian cell line. The polynucleotides encoding the ApoA-I can be codon optimized for expression in recombinant host cells. Preferred host cells are mammalian host cells, including, but not limited, Chinese hamster ovary cells (e.g. CHO-K1; ATCC No. CCL 61; CHO—S (GIBCO Life Technologies Inc., Rockville, MD, Catalog #11619012)), VERO cells, BHK (ATCC No. CRL 1632), BHK 570 (ATCC No. CRL 10314), Hela cells, COS-1 (ATCC No. CRL 1650), COS-7 (ATCC No. CRL 1651), MDCK cells, 293 cells (ATCC No. CRL 1573; Graham et al., J. Gen. Virol. 36:59-72, 1977), 3T3 cells, myeloma cells (especially murine), PC12 cells and W138 cells. In certain embodiments, the mammalian cells, such as CHO—S cells (Invitrogen™, Carlsbad CA), are adapted for growth in serum-free medium. Additional suitable cell lines are known in the art and available from public depositories such as the American Type Culture Collection, Manassas, Va.

In a preferred embodiment, the recombinant ApoA-I is produced by a CHO cell. As the person of ordinary skill in the art will be aware, expression by a mammalian host cell, such as a CHO cell, may undergo post-translational processing (e.g., glycosylation, etc.). The resulting recombinant ApoA-I can have one or more structural features (e.g., glycosylation pattern) that are different from ApoA-I purified from human plasma.

Apomers and Cargomers are described in WO 2019/030575 and WO 2019/030574, respectively, the contents of which are incorporated herein by reference in their entireties.

Lipid binding protein-based complexes such as CER-001 can be formulated for the intended route of administration, for example according to techniques known in the art (e.g., as described in Allen et al., eds., 2012, Remington: The Science and Practice of Pharmacy, 22nd Edition, Pharmaceutical Press, London, UK). CER-001 intended for administration by infusion can be formulated in a phosphate buffer with sucrose and mannitol excipients, for example as described in WO 2012/109162.

Lipid binding protein-based complexes such as CER-001 can be used as drug carriers. See, e.g., WO 2012/109162. Accordingly, in some embodiments, the CER-001 of the disclosure is a carrier for a drug, e.g. a hydrophobic, lipophilic or apolar active agents. Exemplary active agents, include but are not limited to fatty acids, drugs, nucleic acids, vitamins, and/or nutrients. Suitable hydrophobic, lipophilic or apolar active agents are not limited by therapeutic category, and can be, for example, analgesics, anti-inflammatory agents, antihelmimthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarial, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leukotriene inhibitors, macrolides, muscle relaxants, nutritional agents, nucleic acids (e.g., small interfering RNAs), opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Specific, non-limiting examples of suitable hydrophobic, lipophilic, or apolar active agents are: acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. Salts, isomers and derivatives of the above-listed agents may also be used, as well as mixtures.

### 6.1.5. Exemplary Schemes

Exemplary schemes for making sphingomyelins, for example N-palmitoyl-D-erythro-sphingomyelin, and synthetic intermediates useful for making sphingomyelins are presented below. Those skilled in the art will appreciate that variations to the schemes shown below (e.g., the use of alternative solvents, protecting groups, etc.) can be used. In the below schemes, intermediates can be, but are not necessarily, isolated between steps. In some embodiments, one or more of the intermediates shown in the schemes in brackets are not isolated. Thus, in some embodiments, multiple steps of an overall synthesis scheme can be performed as a one-pot synthesis. The halo and bromo intermediates shown in Schemes II(a)-IV(b), although shown at the end or beginning of a scheme, are shown in brackets as the reactions illustrated in the schemes below can be part of a larger overall synthesis scheme wherein the halo and bromo intermediates may not be isolated. For example, an overall synthesis scheme for the synthesis of a sphingomyelin can comprise the reactions shown in Schemes I(a), II(a) and IV(a), the reactions shown in Schemes I(a), III(a) and IV(a), the reactions shown in Schemes I(b), II(b) and IV(b), or the reactions shown in Schemes I(b), III(b) and IV(b). The reactions shown in the schemes can be monitored for reduction or disappearance of starting materials, e.g., by TLC or HPLC.

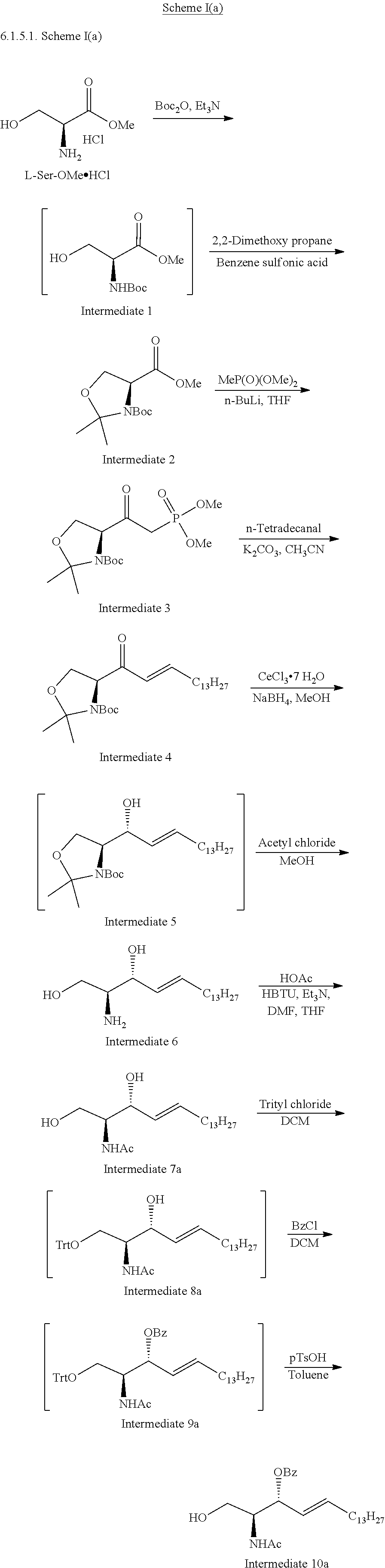

Scheme I(a) shows an exemplary synthesis scheme that can be used to make N-Acyl-3-O-Benzoyl-D-erythro sphingosine (Intermediate 10a). The acyl group can be an acyl group having 3 to 36 carbon atoms and zero to six carbon-carbon double bonds, for example palmitoyl, myristoyl, stearoyl, oleoyl, linoleoyl, linolenoyl, arachidonoyl, or eicosapentaenoyl. Schemes and processes for making Intermediate 10a are additionally described in WO 2014/140787, the contents of which are incorporated herein by reference in their entireties.

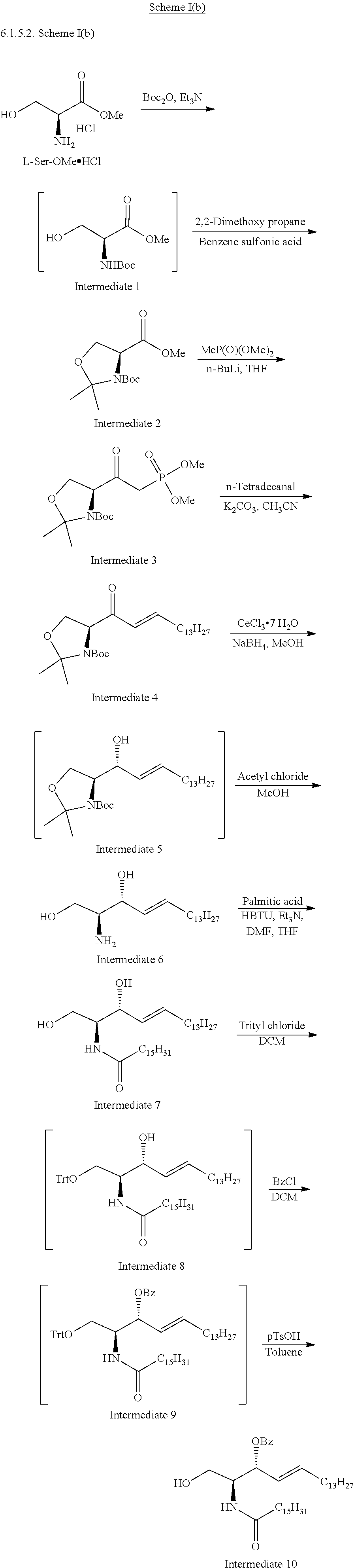

Scheme I(b) shows an exemplary synthesis scheme that can be used to make N-palmitoyl-3-O-Benzoyl-D-erythro sphingosine (Intermediate 10). Scheme I(b) corresponds to Scheme I(a), where the acyl group is palmitoyl.

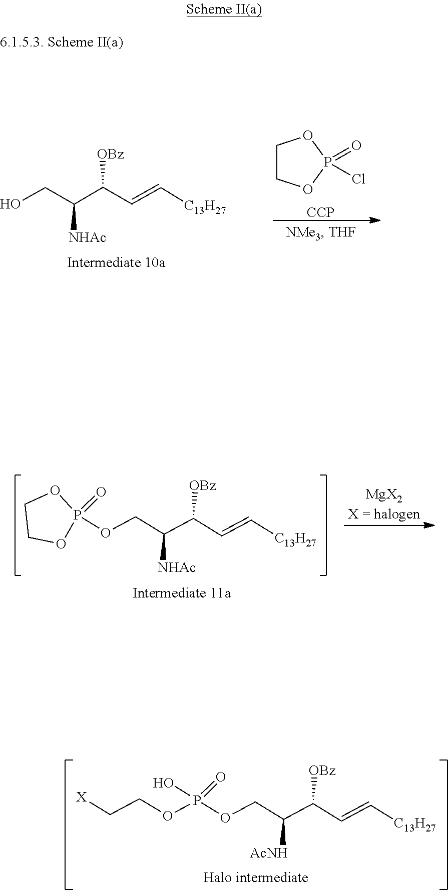

Scheme II(a) shows an exemplary synthesis scheme that can be used to make an exemplary halo intermediate compound of Formula (I) starting from Intermediate 10a. In the scheme, Intermediate 10a is reacted with 2-chloro-2-oxo-1,3,2-dioxaphospholane (CCP) in the presence of trimethylamine (NMe₃) and THF to provide Intermediate 11a. The molar ratio of Intermediate 10a to CCP can be, for example, in a molar ratio range of 3:1 to 1:3 or 2:1 to 1:2, for example 1:1 or 1:1.5. The solution of trimethylamine and THF can be, for example, a 2 M solution of trimethylamine in THF. The molar ratio of Intermediate 10 to trimethylamine can range, for example, from 10:1 to 1:10. In an exemplary embodiment, 5 to 10 equivalents of trimethylamine are used, e.g., 6 equivalents. The reaction can be performed at a suitable temperature, for example at a temperature ranging from −10° C. to 20° C. or 0 to 10° C. The reaction can be monitored by TLC.

Intermediate 11a can be reacted with a magnesium halide, e.g., MgBr₂, to provide the halo intermediate compound of Formula (I). The molar ratio of Intermediate 11s to the magnesium halide can be, for example, in a molar ratio range of 3:1 to 1:3 or 2:1 to 1:2, for example 1:1, 1:1.5, or 1:2. The reaction can be performed at a suitable temperature, for example at a temperature ranging from 20° C. to 40° C., for example 25-35° C. The reaction can be monitored by TLC.

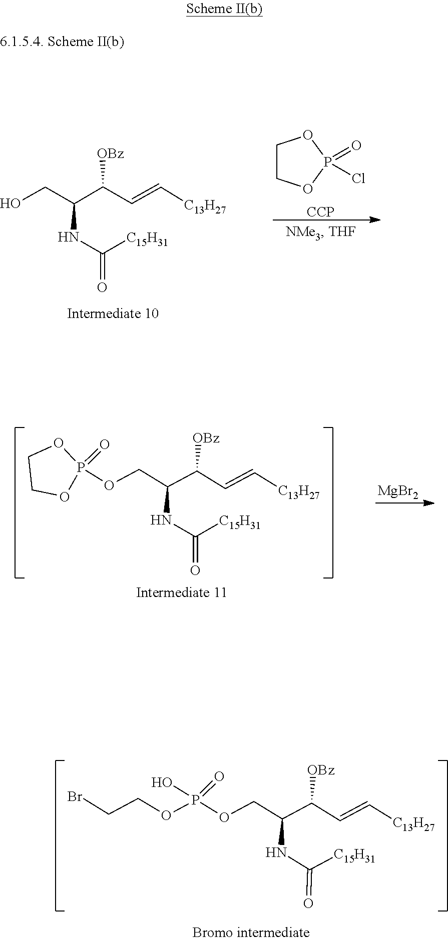

Scheme II(b) shows an exemplary synthesis scheme that can be used to make an exemplary bromo intermediate compound of Formula (I) starting from Intermediate 10. In the scheme, Intermediate 10 is reacted with 2-chloro-2-oxo-1,3,2-dioxaphospholane (CCP) in the presence of trimethylamine (NMe₃) and THF to provide Intermediate 11. The molar ratio of Intermediate 10 to CCP can be, for example, in a molar ratio range of 3:1 to 1:3 or 2:1 to 1:2, for example 1:1 or 1:1.5. The solution of trimethylamine and THF can be, for example, a 2 M solution of trimethylamine in THF. The molar ratio of Intermediate 10 to trimethylamine can range, for example, from 10:1 to 1:10. In an exemplary embodiment, 5 to 10 equivalents of trimethylamine are used, e.g., 6 equivalents. The reaction can be performed at a suitable temperature, for example at a temperature ranging from −10° C. to 20° C. or 0 to 10° C. The reaction can be monitored by TLC.

Intermediate 11 can be reacted with MgBr₂ to provide the bromo intermediate compound of Formula (I). In some embodiments, the MgBr₂ is the form of magnesium bromide ethyl etherate. The molar ratio of Intermediate 11 to MgBr₂ can be, for example, in a molar ratio range of 3:1 to 1:3 or 2:1 to 1:2, for example 1:1, 1:1.5, or 1:2. The reaction can be performed at a suitable temperature, for example at a temperature ranging from 20° C. to 40° C., for example 25-35° C. The reaction can be monitored by TLC.

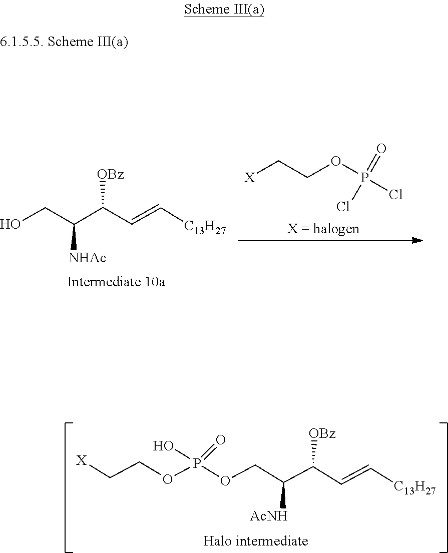

Scheme III(a) shows an exemplary synthesis scheme that can be used to make exemplary halo intermediate compounds of Formula (I) starting from Intermediate 10a. In the scheme, Intermediate 10a is reacted with a 2-haloethyl phosphorodichloridate, e.g., 2-bromoethyl phosphorodichloridate, to provide the halo intermediate compound of Formula (I). The synthesis of the halo intermediate by this route avoids the use of the CCP reagent and any ring opening impurity due to incomplete reaction of Intermediate 11a in Scheme II(a).

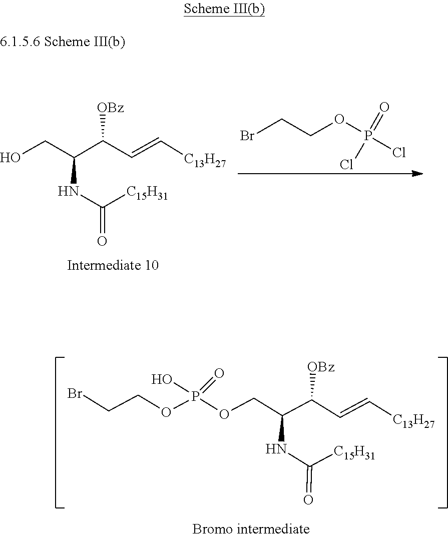

Scheme III(b) shows an exemplary synthesis scheme that can be used to make an exemplary bromo intermediate compound of Formula (I) starting from Intermediate 10. In the scheme, Intermediate 10 is reacted with a 2-bromoethyl phosphorodichloridate to provide the bromo intermediate compound of Formula (I). The synthesis of the bromo intermediate by this route avoids the use of the CCP reagent and any ring opening impurity due to incomplete reaction of Intermediate 11 in Scheme II(b).

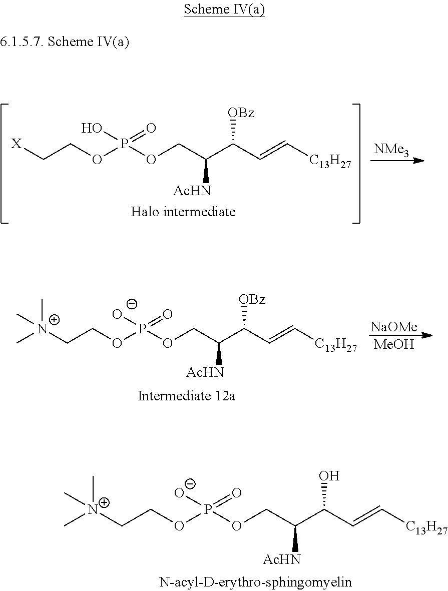

Scheme IV(a) shows an exemplary synthesis scheme for the synthesis of an N-acyl-D-erythro-sphingomyelin starting from a halo intermediate, e.g., a bromo intermediate. The acyl group can be an acyl group having 3 to 36 carbon atoms and zero to six carbon-carbon double bonds, for example palmitoyl, myristoyl, stearoyl, oleoyl, linoleoyl, linolenoyl, arachidonoyl, or eicosapentaenoyl.

The halo intermediate can be reacted with trimethylamine (e.g., liquid) to provide intermediate 12a. The reaction can take place in a suitable solvent, for example methanol or water, at a suitable temperature, for example 30° C. to 60° C. or 40° C. to 50° C. Trimethylamine is preferably used in excess, e.g., 10 to 100 equivalents, 30 to 80 equivalents, or 40 to 60 equivalents, such as 50 equivalents. The reaction can be monitored by TLC.

A workup of Intermediate 12a can be performed before further use. For example, intermediate 12a can be concentrated (e.g., under vacuum below 50° C.), and codistilled with dichloromethane. The residue can be dissolved in a mixture of dichloromethane:methanol (e.g., 2:1, 15 vol) and washed twice with water (e.g., 5 vol). The organic layer can be concentrated to provide Intermediate 12a. If desired, intermediate 12a can be purified, for example by chromatography using 230-400 mesh silica gel with a mixture of dichloromethane and methanol.

Intermediate 12a can be deprotected to provide N-acyl-D-erythro-sphingomyelin, for example by reacting Intermediate 12a with sodium methoxide (NaOMe) in methanol (MeOH) at a suitable temperature, for example a temperature from 10° C. to 40° C. or 20° C. to 30° C., such as 20° C. The reaction can be monitored by HPLC. Exemplary conditions for deprotecting Intermediate 12a are further described in WO 2014/140787.

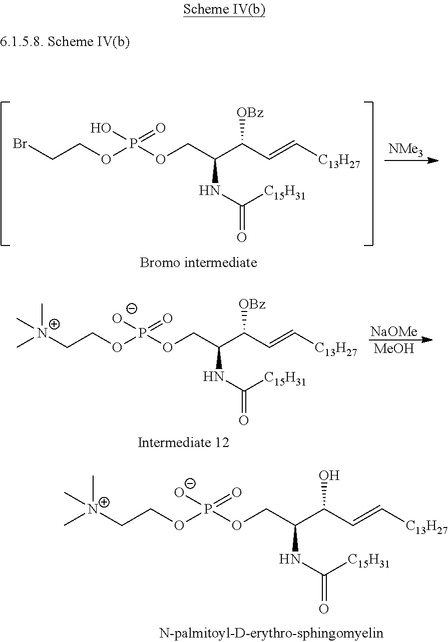

Scheme IV(b) shows an exemplary synthesis scheme for the synthesis of N-palmitoyl-D-erythro-sphingomyelin starting from the bromo intermediate.

The bromo intermediate can be reacted with trimethylamine (e.g., liquid) to provide intermediate 12. The reaction can take place in a suitable solvent, for example methanol or water, at a suitable temperature, for example 30° C. to 60° C. or 40° C. to 50° C. Trimethylamine is preferably used in excess, e.g., 10 to 100 equivalents, 30 to 80 equivalents, or 40 to 60 equivalents, such as 50 equivalents. The reaction can be monitored by TLC.

A workup of Intermediate 12 can be performed before further use. For example, intermediate 12 can be concentrated (e.g., under vacuum below 50° C.), and codistilled with dichloromethane. The residue can be dissolved in a mixture of dichloromethane:methanol (e.g., 2:1, 15 vol) and washed twice with water (e.g., 5 vol). The organic layer can be concentrated to provide Intermediate 12. If desired, intermediate 12 can be purified, for example by chromatography using 230-400 mesh silica gel with a mixture of dichloromethane and methanol

Intermediate 12 can be deprotected to provide N-palmitoyl-D-erythro-sphingomyelin, for example by reacting Intermediate 12 with sodium methoxide (NaOMe) in methanol (MeOH) at a suitable temperature, for example a temperature from 10° C. to 40° C. or 20° C. to 30° C., such as 20° C. The reaction can be monitored by HPLC. Exemplary conditions for deprotecting Intermediate 12a are further described in WO 2014/140787.

### 7. EXAMPLES

### 7.1. Example 1: Synthesis of Sphingomyelin Via Bromo Intermediate

### 7.1.1. Synthesis of N-Palmitoyl-D-erythro-sphingosine (Intermediate 7)

Intermediate 6 (15.0 g, 0.050 mol), HBTU (20.89 g, 0.055 mol), palmitic acid (12.85 g, 0.050 mol) in DMF (105 ml, 7 vol) and THF (360 ml, 24 vol) were charged to a clean and dry round bottom flask (RBF). The reaction mass was cooled to 0 to 5° C. and a solution of triethylamine (13.78 g, 0.136 mol) in THF (15.0 ml, 1.0 vol) was added. The reaction mass was stirred for 3-4 hours. The absence of starting material was confirmed by HPLC. 5% citric acid solution (240 ml, 16.0 vol) was slowly added to the reaction mass and stirred for 2-3 hours at approximately 20° C. The reaction mass was filtered and washed with water. The wet cake was then slurried two times with water at approximately 20° C., filtered and washed with water and acetone. The resulting wet cake was stirred with acetone (420 ml, 28 vol) for 1-2 hours at approximately 20° C., filtered and washed with acetone (75 mL, 5 vol). The isolated solid was dried under vacuum at 30-40° C. for 10-12 hours to yield N-Palmitoyl-D-erythro-sphingosine (Intermediate 7) as a solid product (Output: 14.4 g, Yield: 53.45%).

### 7.1.2. Synthesis of N-Palmitoyl-3-O-benzoyl-D-erythro-sphingosine (Intermediate 10)

Intermediate 7, ethyl acetate (8.0 vol) and triethyl amine (2.0 eq.) were charged to a clean RBF at approximately 20° C. A solution of trityl chloride (1.1 eq.) in ethyl acetate (5 vol) was slowly added and the temperature was raised to 70-80° C. The mixture was stirred for 10 to 12 hours at 70-80° C. A solution of trityl chloride in ethyl acetate (1 vol) was added slowly to the mixture. The mixture was maintained at 70-80° C. for 6-7 hours. The absence of starting material was confirmed by HPLC. The reaction mass was filtered, and the ethyl acetate layer was washed with water. The aqueous layer was extracted with ethyl acetate. The organic layers were wash with saturated NaHCO₃ solution (5 vol). The organic layer was distilled under vacuum, codistilled with dichloromethane to provide intermediate 8 in dichloromethane.

The solution of intermediate 8 in dichloromethane was combined with additional dichloromethane (12 vol), triethyamine (2 eq.), and DMAP (0.1 eq.). The reaction mass was cooled to 0-5° C. Benzoyl chloride (1.5 eq.) was then added to the reaction mass under nitrogen atmosphere. The reaction mass temperature was then raised to approximately 20° C. and stirred for 10-12 hours. The absence of starting material was confirmed by HPLC. Water was added slowing into reaction and the layers were then separated. The aqueous layer was extracted with dichloromethane and combined with the organic layer. The combined organic layers were washed with aqueous sodium bicarbonate solution followed by brine solution. The organic layer was concentrated under vacuum to provide intermediate 9.

Methanol (16 vol) and dichloromethane (16 vol) were added into the crude intermediate 9. The mixture was cooled to 0-10° C. A solution of p-toluene sulfonic acid (0.5 eq.) was slowly added into reaction mass, and the temperature was raised to approximately 20° C. The reaction mass was stirred for 6-10 hours. The absence of starting material was confirmed by HPLC. Triethyl amine (0.25 vol) was added to the reaction mass. The reaction mass was concentrated under vacuum below 45° C., and codistilled two times with methanol (2 vol). The crude intermediate 10 (N-Palmitoyl-3-O-benzoyl-D-erythro-sphingosine) was recrystallized using methanol, cooled to 0 to 5° C. The solid was filtered, washed with methanol, and dried under vacuum at 35-40° C. for 6-10 hours.

The results from three runs are shown in Table 1.

TABLE 1

| | | | | Purity by |
|---|---|---|---|---|
| | Intermediate 7 | Output | Yield (% | HPLC |
| Run | Input (g) | (g) | theoretical) | (% area) |
| 1 | 30.0 | 36.02 | 100.6 | 64.1 |
| 2 | 30.0 | 35.50 | 99.16 | 68.6 |
| 3 | 30.0 | 34.4 | 96.09 | 69.2 |

Crude intermediate 10 was purified by column chromatography using 230-400 mesh silica and a mixture of ethyl acetate and hexanes. The product was eluted in 15-25% ethyl acetate/hexanes. Pure fractions were collected separately and analyzed by TLC/HPLC. Fractions have >90% purity were combined and concentrated under vacuum to provide purified intermediate 10 as solid compound. Table 2 shows yield and purity for three runs.

TABLE 2

| | | | | Purity by |
|---|---|---|---|---|
| | Intermediate 7 | Output | Yield (% | HPLC |
| Run | Input (g) | (g) | theoretical) | (% area) |
| 1 | 25.0 | 10.5 | 42.0 | 94.1 |
| 2 | 25.0 | 9.5 | 38.0 | 94.9 |
| 3 | 25.0 | 10.1 | 40.4 | 95.0 |

### 7.1.3. Synthesis of N-Palmitoyl-3-O-benzoyl-D-erythro-sphingomyelin (Intermediate 12)

Intermediate 10 in THF (15.0 vol) was charged to a clean and dry RBF and cooled to 0 to 10° C. A 2M solution of trimethylamine in THF (6.0 eq.) was added. A solution of 2-chloro-2-oxo-1,3,2-dioxaphospholane (CCP) (1.5 eq.) in acetonitrile (2.5 vol) was slowly added at 0 to 10° C. and allowed to react to provide Intermediate 11. The absence of starting material was monitored by TLC. The reaction mass was cooled to 0-5° C., and magnesium bromide ethyl etherate (2.0 eq.) was added. The temperature of the reaction mass was raised to approximately 20° C., then stirred at 25-35° C. for 12-14 hours under nitrogen atmosphere to provide the bromo intermediate. The absence of intermediate 11 was confirmed by TLC. The reaction mass was concentrated completely under vacuum below 45° C. Methanol (14.0 vol) and THF (7.0 vol) were charged into the residue and the mass was transferred into an autoclave. Precooled (−10 to ~15° C.) trimethylamine (liquid) (50.0 eq.) was charged into the reaction mass and the temperature raised to 40-50° C. The reaction mass was stirred for 24 hours at 40-50° C. The absence of the bromo intermediate was confirmed by TLC. The reaction mass was concentrated completely under vacuum below 50° C., and codistilled with dichloromethane. The residue was dissolved in a mixture of dichloromethane:methanol (2:1, 15 vol) and washed twice with water (5 vol). The organic layer was concentrated to provide Intermediate 12.

The crude Intermediate 12 was purified by chromatography using 230-400 mesh silica gel with a mixture of dichloromethane and methanol. Fractions having >90% purity were combined and concentrated under vacuum. Table 3 shows yield and purity for an exemplary run.

TABLE 3

| | | | Purity by |
|---|---|---|---|
| Intermediate 10 | Output | Yield (% | HPLC |
| Input (g) | (g) | theoretical) | (% area) |
| 7.5 | 6.0 | 63.6 | 91.13% |
| | | | 0.06 RRT: 4.35% |
| | | | 1.14 RRT: 1.28% |

To control the 0.06 RRT impurity, the intermediate 12 was dissolved in dichloromethane:methanol (2:1, 10 vol) and washed with water (5 vol). Table 4 shows the results for an exemplary run.

TABLE 4

| Input | Input | Output Purity | |
|---|---|---|---|
| (g) | purity (%) | by HPLC (%) | Remarks |
| 1.0 | 87.7% 0.06 | 89.8% 0.06 | 0.06 RRT impurity is |
| | RRT: 2.07% | RRT: 0.14% | reduced by methanolic |
| | | | water washing |

Another exemplary run (not subject to washing with methanolic water) was purified on a C18 silica bed using acetonitrile-methanol solvent to provide a purity of greater than 97% (Table 5).

TABLE 5

| | | | Output | |
|---|---|---|---|---|
| Input | Output | Yield | Purity by | |
| (g) | (g) | (%) | HPLC (%) | Remarks |
| 7.5 | 3.5 | 37.1% | 97.9% | Impurity at 0.06 |
| | | | | RRT is 1.25% |

### 7.1.1. Synthesis of N-Palmitoyl-D-erythro-sphingomyelin

Intermediate 12 was charged in methanol (5.0 vol) in a clean and dry RBF. The reaction mass was cooled to 0-5° C. under nitrogen atmosphere. A solution of 30% NaOMe in methanol (0.2 eq.) was added under nitrogen atmosphere, and the temperature was allowed to rise to 25-35° C. The reaction mass was stirred at approximately 20° C. for 10-12 hours. The absence of starting material was confirmed by HPLC. Dichloromethane (10.0 vol) and water (5.0 vol) were charged into the reaction mass and the pH was adjusted to 6-7 by using 1M HCl solution. The layers were separated and the aqueous layer was extracted with dichloromethane (5.0 vol). The organic layer was distilled under vacuum below 35° C., codistilled with methanol (2.4 vol) and dichloromethane (2.4 vol). The residue was dissolved into dichloromethane (2.4 vol) and methanol (2.4 vol), filtered through a micron filter and washed with (1:1) methanol:dichloromethane. The filtrate was distilled completely under vacuum at below 35° C. 1:1 methanol:dichloromethane (12.5 ml, 1 vol) was added, followed by acetone (20 vol) added slowly into the reaction mass. The reaction mass was cooled to 0-5° C. for 5-6 h. The obtained solid was filtered and washed with chilled acetone. The sphingomyelin product was dried under vacuum for 30-50 hours at 30° C. Results for an exemplary run are shown in Table 6.

TABLE 6

| Input of | | | |
|---|---|---|---|
| Intermediate | Output | Yield | Output Purity |
| 12 (g) | (g) | (%) | by HPLC (%) |
| 1.0 | 0.60 | 74.7% | 97.7% |

The impurity at ~0.06 RRT was carried from Intermediate 12, as an impurity at 0.07 RRT of 1.05% was observed. Thus, purity of the final sphingomyelin product can be increased by controlling the 0.06 RRT impurity.

### 7.2. Example 2: Optimization of Intermediate 7 Synthesis

Intermediate 7 is considered a key intermediate in the synthesis of N-Palmitoyl-D-erythro-sphingomyelin. Synthesis of Intermediate involves coupling of palmitic acid and Intermediate 6 by using N,N,N′,N′-Tetramethyl-O-(1H-benzotriazol-1-yl) uronium hexafluorophosphate (HBTU) reagent in the presence of triethylamine as a base. The mole equivalents of trimethylamine and HBTU were optimized.

To evaluate the impact of different mole equivalent of triethylamine on the Intermediate 7 reaction, reactions were performed using different mole equivalent of triethylamine. Results are shown in Table 7.

TABLE 7

| | Input | | | |
|---|---|---|---|---|
| | Intermediate | Mole equiv. | Yield | Purity |
| Study | 6 (g) | of triethylamine | (%) | (%) |
| 1 | 1.0 | 2.72 | 55.68 | 88.0 |
| 2 | 2.0 | 2.0 | 65.85 | 94.05 |
| 3 | 2.0 | 3.0 | 64.07 | 94.48 |
| 4 | 15 | 2.72 | 49.0 | 97.08 |

There was no observed significant impact of lower and higher equivalents of triethylamine on Intermediate 7 production.

HBTU activates the palmitic acid in the Intermediate 7 reaction. To evaluate the impact of different mole equivalent of HBTU on the Intermediate 7 reaction, reactions were performed using different mole equivalent of HBTU. Results are shown in Table 8.

TABLE 8

| | Input | | | |
|---|---|---|---|---|
| | Intermediate | Mole equiv. | Yield | Purity |
| Study | 6 (g) | of triethylamine | (%) | (%) |
| 1 | 1.0 | 1.1 | 55.68 | 88.0 |
| 2 | 1.0 | 1.3 | 50.67 | 92.21 |
| 3 | 2.0 | 1.0 | 58.50 | 90.72 |
| 4 | 15.0 | 1.1 | 49.0 | 97.08 |

There was no observed significant impact of lower and higher equivalents of HBTU on Intermediate 7 production.

### 7.3. Example 3: Characterization of Synthetic Sphingomyelin

Synthetic sphingomyelin (sSph) produced according to the processes described in Example 1 was characterized and compared to sphingomyelin derived from egg (eggSph) (NOF Corporation).

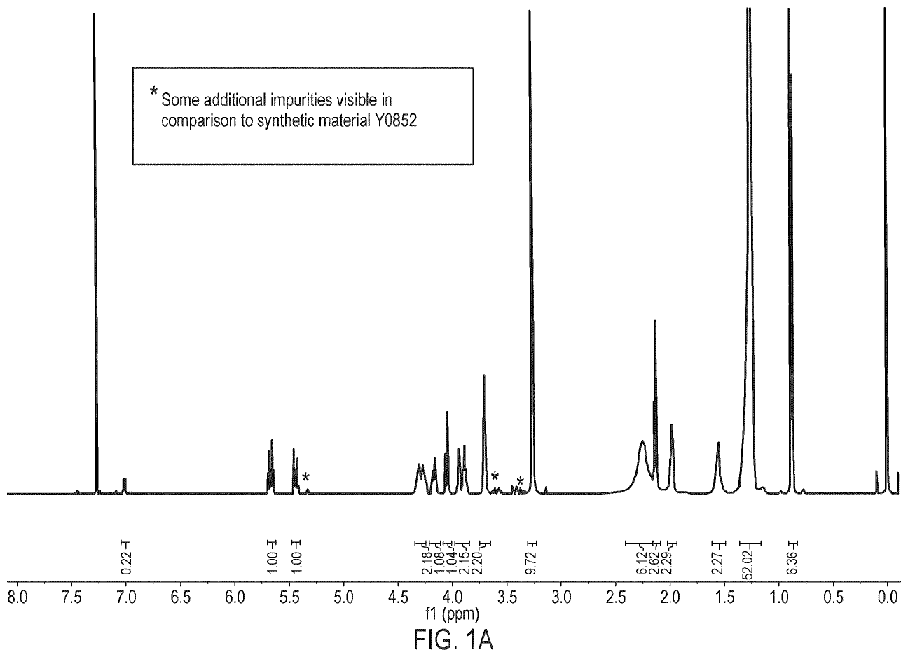

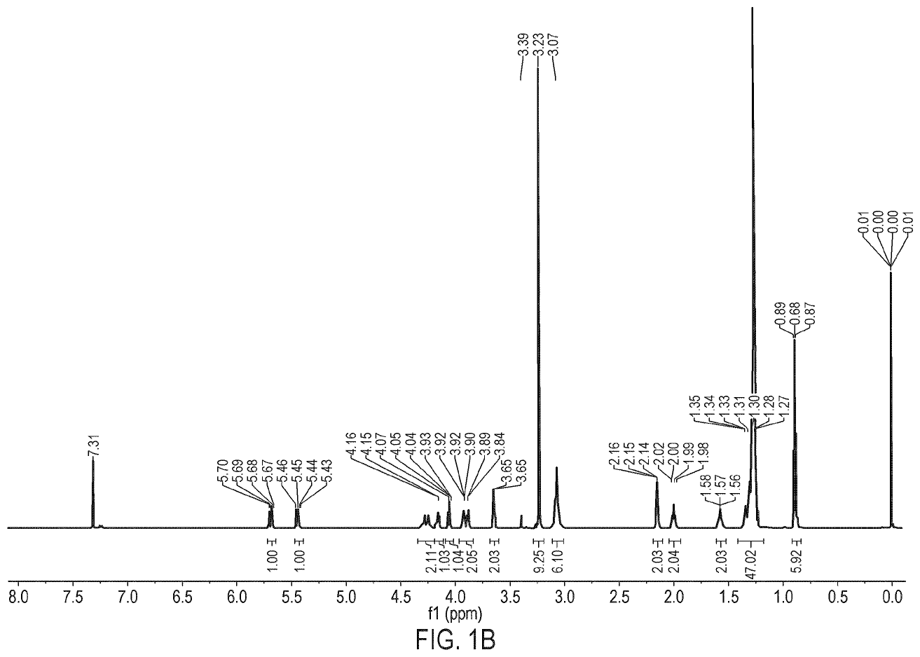

A ¹H-NMR spectrum of eggSph is shown in FIG. 1A and a 1H-NMR spectrum of sSph is shown in FIG. 1B. The spectra show that eggSph has some additional impurities not observed in the sSph.

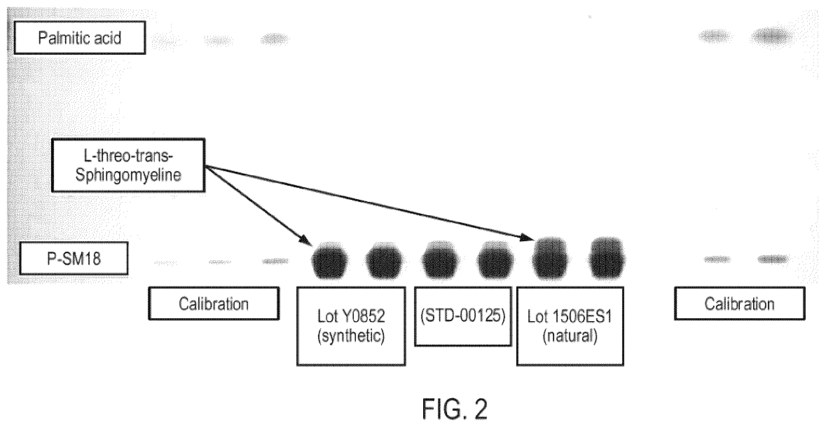

High-performance thin-layer chromatography (HPTLC) data for egg Sph and sSph is shown in Table 9 and FIG. 2.

TABLE 9

| R_{f} | rR_{f} relative | | |
|---|---|---|---|
| Retardation | Retardation | eggSph | sSph |
| Factor | Factor | % w/w | % w/w |
| 0.15 | 0.65 | 0.08% | n.d. |
| 0.25/027 | 1.17/1.25 | >10% | 0.6% |
| 0.39 | 1.95 | n.d. | <0.05% |
| 0.48 | 2.40 | n.d. | <0.05% |
| 0.73 | 3.65 | n.d. | <0.05% |

| | | |
|---|---|---|
| Total Purity | <89.9% | 99.4% |

### 7.4. Example 4: Comparison of CER-001 Made with Egg Sphingomyelin with CER-001 Made with Synthetic Sphingomyelin

### 7.4.1. Cholesterol Mobilization in Rabbits

Pharmacology studies were conducted to compare CER-001 made with eggSph with CER-001 made with sSph produced according to the process of Example 1. Preparations were evaluated in in vivo cholesterol mobilization studies performed in rabbits.

In this model, increases in plasma or HDL cholesterol levels are indicative of tissue cholesterol mobilization and transfer to HDL. CER-001 made with eggSph and CER-001 made with sSph were infused into fasted rabbits at doses of 5 mg/kg or 20 mg/kg and compared to a vehicle group. There were 4 animals per group. At various times post dose, plasma phospholipid, plasma human ApoA-I levels, plasma HDL, plasma total cholesterol were measured. Baseline values were subtracted to determine the increase in all the parameters at the exception of ApoA-I. At 30-34 hours post dose these parameters had returned to baseline.

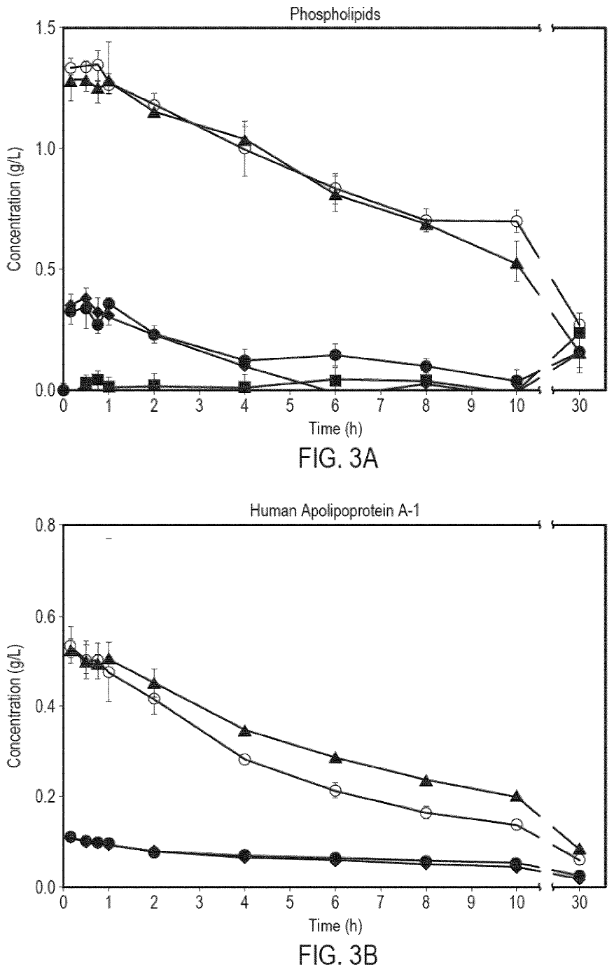

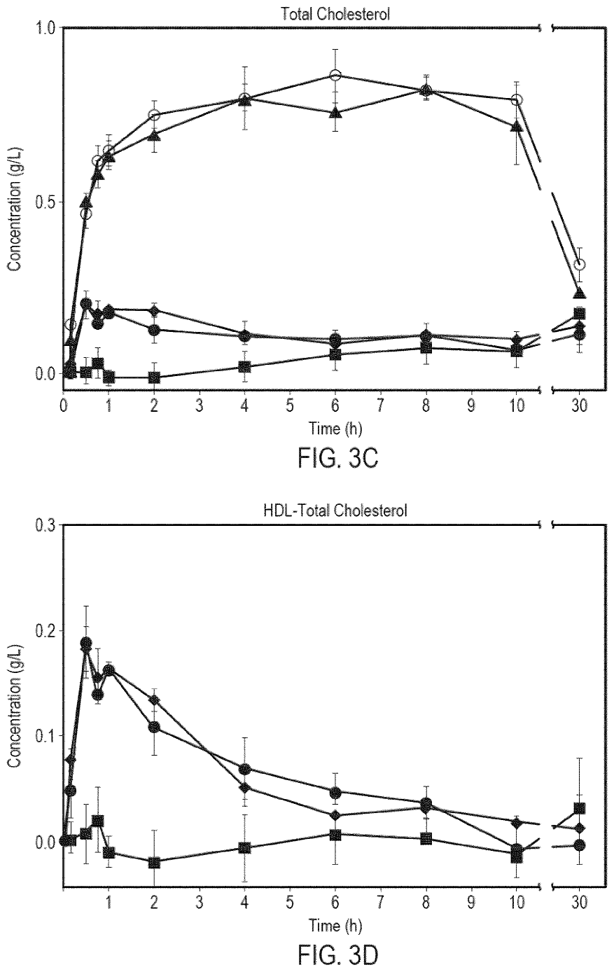

When the different CER-001 complexes were infused into rabbits at doses of 5 and 20 mg/kg, there was a substantially identical and dose-dependent increase in plasma phospholipids and human ApoA-I at the doses tested (FIG. 3A and FIG. 3B). This reflected the infusion of the human ApoA-I and phospholipid containing CER-001 complexes and suggests that these CER-001 preparations were stable in the circulation and caused cholesterol mobilization. A similar and dose-dependent increase in cholesterol mobilization was also observed as both the total plasma cholesterol and total HDL cholesterol levels increased (FIG. 3C and FIG. 3D).

### 7.4.2. Cholesterol Efflux Assay

The biological Potency of CER-001 batches was measured using a Fu5AH rat hepatoma cell-based cholesterol efflux assay. The Fu5AH rat hepatoma cells have high expression of the scavenger receptor class B type I (SRB1), which facilitate the bidirectional flux of cholesterol between the cells and mature HDL. The method was selected based on the specificity of the HDL-mediated cholesterol efflux activity in this experimental model.

Fu5AH cells were labeled with 3H-cholesterol for 24 hours. Acceptor media for efflux was prepared for each CER-001 sample (at 30, 20 and 10 μg/ml, diluted with MEM buffered with 25 mM HEPES) and for controls, including ApoA-I purified from human plasma (20 μg/mL); HDL3, 2% human serum and medium alone. Acceptor media containing samples and controls were added to the cells for 4 hours and efflux media and cell monolayers was assessed to determine the percent of cholesterol released from the Fu5AH cells. Biological activity of each test article is expressed as percent of cholesterol efflux relative to CER-001 Reference Standard, used as an experimental control at the same concentration as the test articles.

Table 10 shows biological activity data of CER-001 made using eggSph and trials using sSph synthesized as in Example 1. Final product quality of CER-001 data of representative process trials using sSph are compiled in Table 5 and compared with representative production batches of CER-001 using Sph derived from eggs. These data show comparability of the materials.

TABLE 10

| | | CER-001 Batch | | | | | |
|---|---|---|---|---|---|---|---|
| | Acceptance | 1 (egg | 2 (egg | 3 (egg | 4 (egg | | |
| Method | Criterion | Sph) | Sph) | Sph) | Sph) | 5 (sSph) | 6 (sSph) |
| Appearance | Clear to translucent, | Complies | Complies | Complies | Complies | Complies | Complies |
| | free from visible | | | | | | |
| | particles | | | | | | |
| pH | 6.0-9.0 | 7.3 | 7.6 | 7.4 | 7.5 | 7.7 | NP |
| Osmolality | 220-380 mOsm/kg | 273 | 271 | 271 | 272 | 294 | NP |
| Identity | Rtₛₐₘₚₗₑ = Rt_{standard} ± | Complies | Complies | Complies | Complies | Complies | Complies |
| CER-001 | 5% | | | | | | |
| Complex by | | | | | | | |
| GPC | | | | | | | |
| Identity | Rtₛₐₘₚₗₑ = Rt_{standard} ± | Complies | Complies | Complies | Complies | Complies | Complies |
| ApoA-I by | 5% | | | | | | |
| HP-SEC | | | | | | | |
| Content | 8.0 mg/mL ± 20% | 8.6 | 7.7 | 8.3 | 7.6 | 7.4 | 8.0 |
| ApoA-I by | | | | | | | |
| HP-SEC | | | | | | | |
| Content | 21.0 mg/mL ± 20% | 23.3 | 21.2 | 21.6 | 21.0 | 21.2 | NP |
| Sph by | | | | | | | |
| Enzymatic | | | | | | | |
| Assay | | | | | | | |
| Purity CER- | NLT 80% CER-001 | 95% | 95% | 94% | 99% | 99% | 94% |
| 001 Complexes | | | | | | | |
| by GPC | Complexes | | | | | | |
| Purity | NLT 85% | 99% | 98% | 98% | 98% | NP | NP |
| ApoA-I by | | | | | | | |
| SDS-PAGE | | | | | | | |
| Purity | Avg. dia. NMT 20 | 12 | 13 | 13 | 14 | NP | 13 |
| Particle | nm | 3 | 3 | 4 | 5 | | 5 |
| Size | Peak width NMT 10 | | | | | | |
| Distribution | nm | | | | | | |
| by DLS | | | | | | | |
| LPO | NMT 2% mol/mol | <0.01% | <0.01% | <0.01% | <0.01% | NP | NP |
| | Sph | | | | | | |
| Biological | Report results, % of | 117% | 91% | 100% | 77% | 92% | NP |
| Potency by | Reference Standard | | | | | | |
| Fu5AH | | | | | | | |
| cholesterol | | | | | | | |
| efflux | | | | | | | |

NP = not performed

### 7.4.3. CER-001 Complex Identity, Size, Purity and Particle Size Distribution

CER-001 made with egg Sph or sSph were evaluated by gel permeation chromatography (GPC). This method allows for resolution of the CER-001 complexes from free protein (ApoA-I) and phospholipids (Sph and DPPG). A Tosoh TSK-GEL G3000SW_{XL} was used. Samples, including CER-001 Reference Standard, were prepared by dilution with mobile phase.

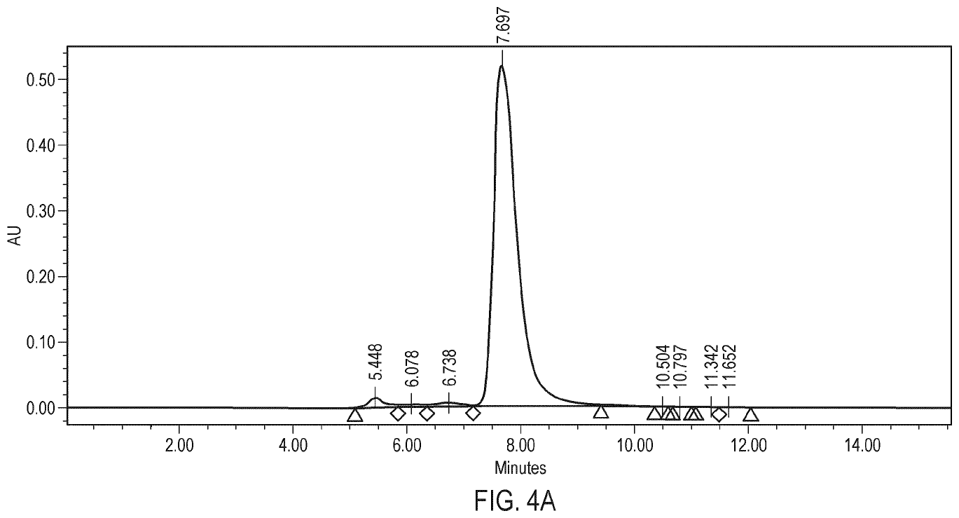

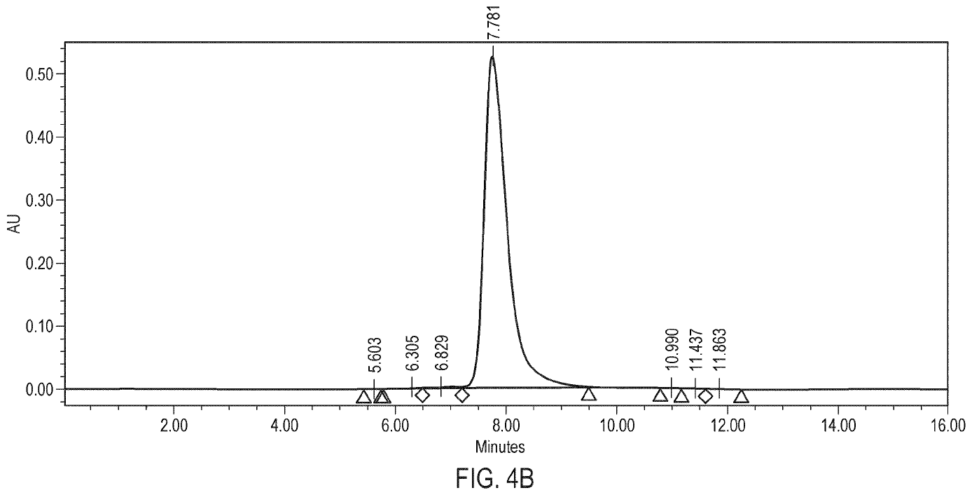

A comparison of the GPC profiles of CER-001 from eggSph reference batch to a sSph batch are shown in FIGS. 4A-4B. The profiles indicate that the complexes made with sSph are about the same size and of comparable quality as the complexes made with egg Sph.

The average particle size and size distribution of CER-001 complexes was monitored by Dynamic Light Scattering (DLS). DLS measures the speed of the movement of the CER-001 particles, which is correlated to the time dependency of the fluctuations of the intensity of laser light scattered from the particles as they diffuse in solution. DLS analysis was performed using a Malvern Zetasizer Nano ZS. Particle Size Distribution of the tested CER-001 batches were comparable (see Table 10).

### 8. SPECIFIC EMBODIMENTS

The present disclosure is exemplified by the specific embodiments below.
1. A compound of Formula (I):

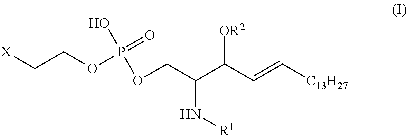

or a salt thereof, wherein:
X is halogen;
R¹ is an acyl group having 3 to 36 carbon atoms and zero to six carbon-carbon double bonds; and
R² is a protecting group.
2. The compound of embodiment 1, which is a compound of Formula (Ia):

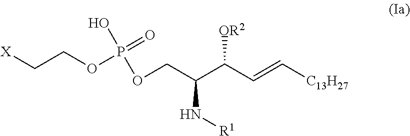

or a salt thereof.
3. The compound of embodiment 1 or embodiment 2, or a salt thereof, wherein X is Br.
4. The compound of any one of embodiments 1 to 3, or a salt thereof, wherein R¹ is palmitoyl, myristoyl, stearoyl, oleoyl, linoleoyl, linolenoyl, arachidonoyl, or eicosapentaenoyl.
5. The compound of embodiment 4 or a salt thereof, wherein R¹ is palmitoyl.
6. The compound of any one of embodiments 1 to 5, or a salt thereof, wherein R² is a benzoyl group.
7. The compound of embodiment 1, which is

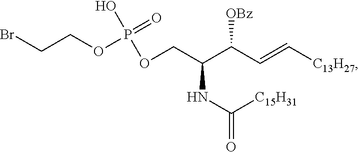

or a salt thereof.
8. A process for synthesizing a sphingomyelin or a salt thereof, comprising:
   a) reacting a compound of Formula (I)

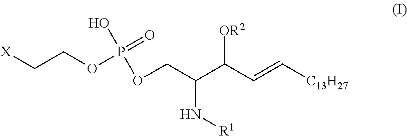

or a salt thereof with trimethylamine to produce a compound of Formula (II)

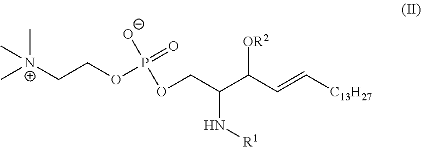

or a salt thereof, wherein
X is halogen;
R¹ is an acyl group having 3 to 36 carbon atoms and zero to six carbon-carbon double bonds; and
R² is a protecting group;
and
   b) removing the R² protecting group from the compound of Formula (II) or a salt thereof to produce a sphingomyelin of Formula (III):

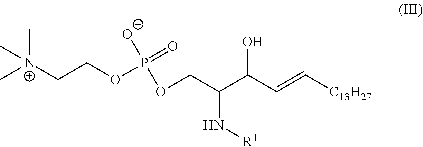

or a salt thereof, wherein R¹ is as defined step (a).
9. The process of embodiment 8, wherein step (a) comprises reacting a compound of Formula (Ia)

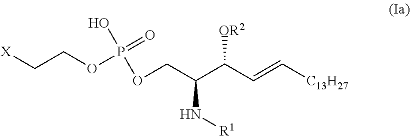

or a salt thereof with trimethylamine to produce a compound of Formula (IIa)

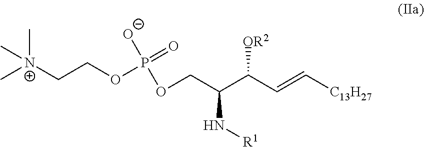

or a salt thereof.
10. The process of embodiment 8 or embodiment 9, wherein X is Br.
11. The process of any one of embodiments 8 to 10, wherein R¹ is palmitoyl, myristoyl, stearoyl, oleoyl, linoleoyl, linolenoyl, arachidonoyl, or eicosapentaenoyl.
12. The process of embodiment 11, wherein R¹ is palmitoyl.
13. The process of any one of embodiments 8 to 12, wherein R² is a benzoyl group.
14. The process of any one of embodiments 8 to 13, wherein the trimethylamine is liquid.
15. The process of any one of embodiments 8 to 14, wherein step (a) comprises reacting the compound of Formula (I) or a salt thereof with trimethylamine in methanol.
16. The process of any one of embodiments 8 to 14, wherein step (a) comprises reacting the compound of Formula (I) or a salt thereof with trimethylamine in a mixture of methanol and tetrahydrofuran (THF), optionally wherein the mixture is a 2:1 mixture of methanol and THF.
17. The process of any one of embodiments 8 to 14, wherein step (a) comprises reacting the compound of Formula (I) or a salt thereof with trimethylamine in water.
18. The process of any one of embodiments 8 to 17, wherein in step (a) 20 to 100 moles of trimethylamine are used per mole of compound of Formula (I) or salt thereof, optionally wherein 30 to 80 moles of trimethylamine are used per mole of compound of Formula (I) or salt thereof, optionally wherein 40 to 60 moles of trimethylamine are used per mole of compound of Formula (I) or salt thereof, optionally wherein 50 moles of trimethylamine are used per mole of compound of Formula (I) or salt thereof.
19. The process of any one of embodiments 8 to 18, wherein step (a) is performed at a temperature of 30° C. to 60° C., optionally 40° C. to 50° C.
20. The process of any one of embodiments 8 to 19, wherein the reaction of step (a) is monitored by thin layer chromatography (TLC).
21. The process of any one of embodiments 8 to 20, wherein the reaction of step (a) is allowed to proceed to completion.
22. The process of any one of embodiments 8 to 21, wherein the reaction of step (a) is allowed to proceed for 18 to 30 hours, optionally 22 to 26 hours.
23. The process of any one of embodiments 8 to 22, wherein step (b) comprises reacting the compound of Formula (II) or a salt thereof with a base.
24. The process of embodiment 23, wherein the base is sodium methoxide, potassium carbonate, or lithium hydroxide.
25. The process of embodiment 24, wherein the base is sodium methoxide.
26. The process of any one of embodiments 23 to 25, wherein step (b) is performed in a protic polar solvent, optionally which is methanol, ethanol, n-propanol, isopropanol, or a mixture of any of the foregoing.
27. The process of embodiment 26, wherein the protic polar solvent is methanol.
28. The process of any one of embodiments 8 to 27, wherein the compound of Formula (I) or salt thereof is the product of a process comprising reacting a compound of Formula (0a)

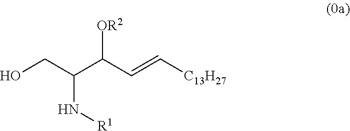

or a salt thereof with

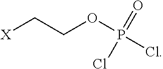

29. The process of any one of embodiments 8 to 27, wherein the compound of Formula (I) or salt thereof is the product of a process comprising reacting a compound of Formula (0c)

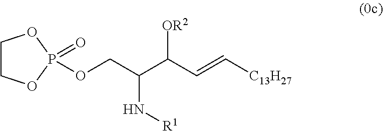

or a salt thereof with MgX₂.
30. The process of any one of embodiments 8 to 29, further comprising isolating and/or purifying the sphingomyelin or a salt thereof.
31. Sphingomyelin or salt thereof produced by the process of any one of embodiments 8 to 30.
32. The sphingomyelin or a salt thereof of embodiment 31, having a purity of at least 99% as measured by high performance thin layer chromatography.
33. A process for synthesizing a protected sphingomyelin or a salt thereof, comprising reacting a compound of Formula (I)

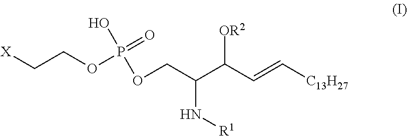

or a salt thereof with trimethylamine to produce a compound of Formula (II)

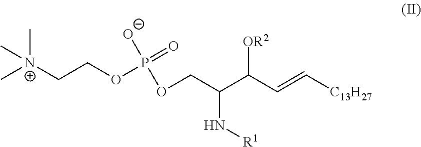

or a salt thereof, wherein
X is halogen;
R¹ is an acyl group having 3 to 36 carbon atoms and zero to six carbon-carbon double bonds; and
R² is a protecting group.
34. The process of embodiment 33, which comprises reacting a compound of Formula (Ia)

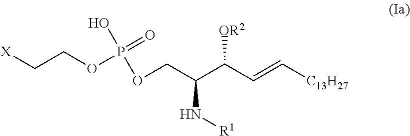

or a salt thereof with trimethylamine to produce a compound of Formula (IIa)

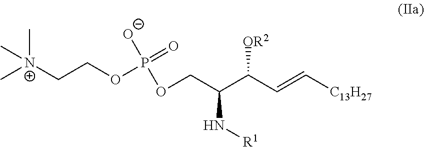

or a salt thereof.
35. The process of embodiment 33 or embodiment 34, wherein X is Br.
36. The process of any one of embodiments 33 to 35, wherein R¹ is palmitoyl, myristoyl, stearoyl, oleoyl, linoleoyl, linolenoyl, arachidonoyl, or eicosapentaenoyl.
37. The process of embodiment 36, wherein R¹ is palmitoyl.
38. The process of any one of embodiments 33 to 37, wherein R² is a benzoyl group.
39. The process of any one of embodiments 33 to 38, wherein the trimethylamine is liquid.
40. The process of any one of embodiments 33 to 39, which comprises reacting the compound of Formula (I) or a salt thereof with trimethylamine in methanol.
41. The process of any one of embodiments 33 to 39, which comprises reacting the compound of Formula (I) or a salt thereof with trimethylamine in a mixture of methanol and tetrahydrofuran (THF).
42. The process of any one of embodiments 33 to 39, which comprises reacting the compound of Formula (I) or a salt thereof with trimethylamine in water.
43. The process of any one of embodiments 33 to 42, wherein 20 to 100 moles of trimethylamine are used per mole of compound of Formula (I) or salt thereof, optionally wherein 30 to 80 moles of trimethylamine are used per mole of compound of Formula (I) or salt thereof, optionally wherein 40 to 60 moles of trimethylamine are used per mole of compound of Formula (I) or salt thereof, optionally wherein 50 moles of trimethylamine are used per mole of compound of Formula (I) or salt thereof.
44. The process of any one of embodiments 33 to 43, wherein the reaction is performed at a temperature of 30° C. to 60° C., optionally 40° C. to 50° C.
45. The process of any one of embodiments 33 to 44, wherein the reaction is monitored by thin layer chromatography (TLC).
46. The process of any one of embodiments 33 to 45, wherein the reaction is allowed to proceed to completion.
47. The process of any one of embodiments 33 to 46, wherein the reaction is allowed to proceed for 18 to 30 hours, optionally 22 to 26 hours.
48. The process of any one of embodiments 33 to 47, wherein the compound of Formula (I) or salt thereof is the product of a process comprising reacting a compound of Formula (0a)

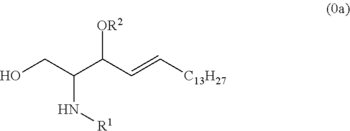

or a salt thereof with

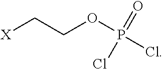

49. The process of any one of embodiments 33 to 47, wherein the compound of Formula (I) or salt thereof is the product of a process comprising reacting a compound of Formula (0c)

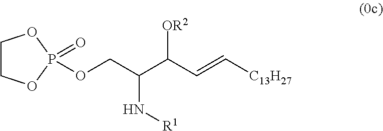

or a salt thereof with MgX₂.
50. A process for making a compound of Formula (I):

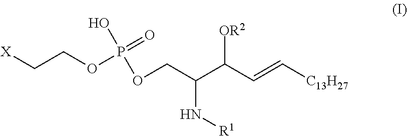

or a salt thereof, wherein:
   X is halogen;
   R¹ is an acyl group having 3 to 36 carbon atoms and zero to six carbon-carbon double bonds; and
   R² is a protecting group,
the process comprising reacting a compound of Formula (0a)

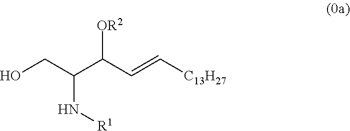

or a salt thereof with

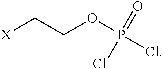

51. The process of embodiment 50, which comprises reacting a compound of Formula (0b)

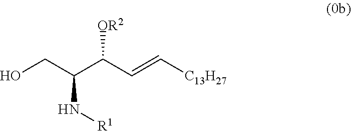

or a salt thereof with

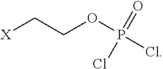

52. The process of any one of embodiments 50 to 51, wherein X is Br.
53. The process of any one of embodiments 50 to 52, wherein R¹ is palmitoyl, myristoyl, stearoyl, oleoyl, linoleoyl, linolenoyl, arachidonoyl, or eicosapentaenoyl.
54. The process of embodiment 53, wherein R¹ is palmitoyl.
55. The process of any one of embodiments 50 to 54, wherein R² is a benzoyl group.
56. A process for making a compound of Formula (I):

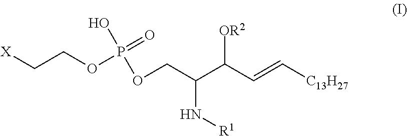

or a salt thereof, wherein:
X is halogen;
R¹ is an acyl group having 3 to 36 carbon atoms and zero to six carbon-carbon double bonds; and
R² is a protecting group,
   the process comprising reacting a compound of Formula (0c)

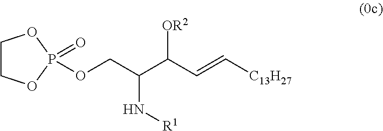

or a salt thereof with MgX₂.
57. The process of embodiment 56, which comprises reacting a compound of Formula (0d)

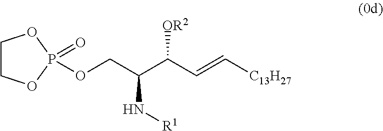

or a salt thereof with MgX₂.
58. The process of any one of embodiments 56 to 57, wherein X is Br.
59. The process of any one of embodiments 56 to 58, wherein R¹ is palmitoyl, myristoyl, stearoyl, oleoyl, linoleoyl, linolenoyl, arachidonoyl, or eicosapentaenoyl.
60. The process of embodiment 59, wherein R¹ is palmitoyl.
61. The process of any one of embodiments 56 to 60, wherein R² is a benzoyl group.
62. The process of any one of embodiments 57 to 61, wherein the molar ratio of the compound of Formula (0c) to MgX₂ is 1:1 to 1:3, 2:1 to 1:2, optionally 1:1, 1:1.5, or 1:2.
63. The process of any one of embodiments 57 to 62, wherein the reaction is performed at a temperature of 20° C. to 40° C., optionally 25 to 35° C.
64. The process of any one of embodiments 56 to 63, wherein the compound of Formula (0c) is the product of a process comprising reacting

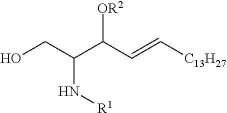

with 2-chloro-2-oxo-1,3,2-dioxaphospholane (CCP).
65. The process of embodiment 64, wherein molar ratio of

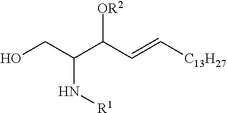

to CCP in the reaction to form the compound of Formula (0c) is 3:1 to 1:3, optionally 2:1 to 1:2, optionally 1:1 or 1:1.5.
66. The process of embodiment 64 or embodiment 65, wherein the reaction to form the compound of Formula (0c) is performed at 10° C. to 20° C. or 0 to 10° C.
67. A lipid binding protein-based complex comprising the sphingomyelin or salt thereof of embodiment 31 or embodiment 32.
68. The lipid binding protein-based complex of embodiment 67, which is CER-001.
69. The lipid binding protein-based complex of embodiment 67 or embodiment 68, which is a carrier for a drug.
70. A pharmaceutical composition comprising the lipid binding protein-based complex of any one of embodiments 65 to 69 and a pharmaceutically acceptable excipient.
71. A process for making a lipid binding protein-based complex comprising combining the sphingomyelin or salt thereof of embodiment 31 or embodiment 32 with a lipid binding protein.
72. The process of embodiment 71, which comprises combining the sphingomyelin or salt thereof and the lipid binding protein to form a mixture and, subsequently, thermal cycling the mixture.
73. The process of embodiment 71 or 72, wherein the lipid binding protein is apolipoprotein A-I (ApoA-I).
74. The process of embodiment 73, wherein the ApoA-I is recombinant.
75. The process of embodiment 73 or embodiment 74, wherein the amino acid sequence of the ApoA-I comprises amino acids 25 to 267 of SEQ ID NO:1.
76. The process of any one of embodiments 73 to 75, wherein the ApoA-I is pre-complexed with DPPG prior to combining with the sphingomyelin or salt thereof.
77. The process of embodiment 76, wherein the weight ratio of ApoA-I to lipid is 1:2.7.
78. The process of embodiment 76 or embodiment 77, wherein the sphingomyelin to DPPG weight ratio is 97:3.
79. The process of any one of embodiments 76 to 78, which comprises combining the ApoA-I pre-complexed with DPPG with the sphingomyelin or salt thereof to form an ApoA-I/DPPG/sphingomyelin mixture and thermal cycling the ApoA-I/DPPG/sphingomyelin mixture between 57° C.±10° C. and 37° C.±10° C.
80. The process of any one of embodiments 76 to 78, which comprises combining the ApoA-I pre-complexed with DPPG with the sphingomyelin or salt thereof to form an ApoA-I/DPPG/sphingomyelin mixture and thermal cycling the ApoA-I/DPPG/sphingomyelin mixture between 57° C.±5° C. and 37° C.±5° C.
81. The process of any one of embodiments 76 to 78, which comprises combining the ApoA-I pre-complexed with DPPG with the sphingomyelin or salt thereof to form an ApoA-I/DPPG/sphingomyelin mixture and thermal cycling the ApoA-I/DPPG/sphingomyelin mixture between 57° C.±2° C. and 37° C.±2° C.
82. The process of any one of embodiments 76 to 78, which comprises combining the ApoA-I pre-complexed with DPPG with the sphingomyelin or salt thereof to form an ApoA-I/DPPG/sphingomyelin mixture and thermal cycling the ApoA-I/DPPG/sphingomyelin mixture between 57° C. and 37° C.
83. The process of any one of embodiments 79 to 82, which comprises thermal cycling the ApoA-I/DPPG/sphingomyelin mixture until complexes at least 98%, or at least 99% homogeneous, as reflected by a single peak in gel permeation chromatography, are formed.
84. A lipid binding protein-based complex obtained or obtainable by the process of any one of embodiments 71 to 83.
85. The process of embodiment 74, wherein the ApoA-I is produced by a mammalian host cell.
86. The process of embodiment 85, wherein the mammalian host cell is a CHO cell.
87. The lipid binding protein-based complex of embodiment 68, wherein the ApoA-I is recombinant and is produced by a mammalian host cell.
88. The lipid binding protein-based complex of embodiment 87, wherein the ApoA-I comprises amino acids 25 to 267 of SEQ ID NO:1.
89. The lipid binding protein-based complex of embodiment 87 or embodiment 88, wherein the mammalian host cell is a CHO cell.
90. The lipid binding protein-based complex of any one of embodiments 87 to 89, wherein the ApoA-I has undergone post-translational processing (e.g., glycosylation) such that the ApoA-I has one or more structural features (e.g., glycosylation pattern) that are different from human ApoA-I purified from human plasma.
91. An Apolipoprotein A-I (“ApoA-I”) formulation comprising ApoA-I and the sphingomyelin or salt thereof of embodiment 31 or embodiment 32.
92. The ApoA-I formulation of embodiment 91, further comprising DPPG, optionally wherein the formulation has a 1:2.7 Apo-AI weight:total lipid weight ratio and a SM:DPPG weight:weight ratio of 97:3.
93. The ApoA-I formulation of embodiment 91 or 92, wherein the ApoA-I is produced by a mammalian host cell.
94. The ApoA-I formulation of embodiment 93, wherein the mammalian host cell is a CHO cell.
95. The ApoA-I formulation of any one of embodiments 91 to 94, wherein the ApoA-I comprises amino acids 25 to 267 of SEQ ID NO:1.

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A compound of Formula (I): 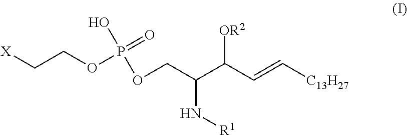
or a salt thereof, wherein:
X is halogen;
R¹ is an acyl group having 3 to 36 carbon atoms and zero to six carbon-carbon double bonds; and
R² is a protecting group.

2. The compound of claim 1, which is a compound of Formula (Ia): 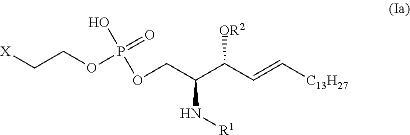
or a salt thereof.

3. The compound of claim 1 or claim 2, or a salt thereof, wherein X is Br; R¹ is palmitoyl, myristoyl, stearoyl, oleoyl, linoleoyl, linolenoyl, arachidonoyl, or eicosapentaenoyl, preferably palimitoyl; and/or R² is a benzoyl group.

4. The compound of claim 1, which is 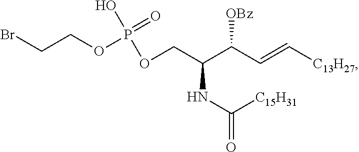
or a salt thereof.

5. A process for synthesizing a sphingomyelin or a salt thereof, comprising:
a) reacting a compound of Formula (I) 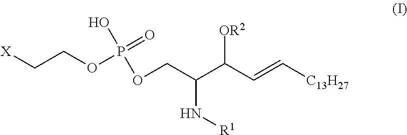
or a salt thereof with trimethylamine optionally wherein the trimethylamine is liquid, to produce a compound of Formula (II) 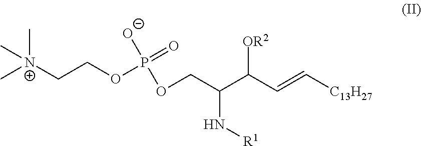
or a salt thereof, wherein
X is halogen;
R¹ is an acyl group having 3 to 36 carbon atoms and zero to six carbon-carbon double bonds; and
R² is a protecting group;
and
b) removing the R² protecting group from the compound of Formula (II) or a salt thereof to produce a sphingomyelin of Formula (III): 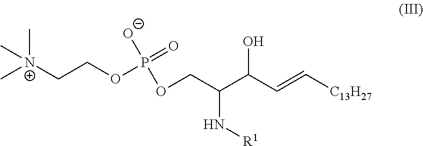
or a salt thereof, wherein R¹ is as defined in step (a).

6. The process of claim 5, wherein step (a) comprises reacting a compound of Formula (Ia) 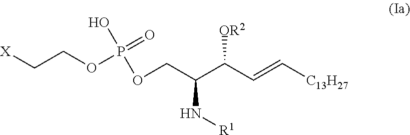
or a salt thereof with trimethylamine, optionally wherein the trimethylamine is liquid, to produce a compound of Formula (IIa) 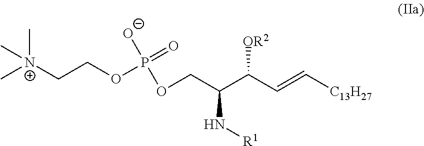
or a salt thereof.

7. The process of claim 5 or claim 6, wherein X is Br; R¹ is palmitoyl, myristoyl, stearoyl, oleoyl, linoleoyl, linolenoyl, arachidonoyl, or eicosapentaenoyl, preferably palmitoyl; and/or R² is a benzoyl group.

8. The process of any one of claims 5 to 7, wherein step (a) comprises reacting the compound of Formula (I) or a salt thereof with trimethylamine (i) in methanol; (ii) in a mixture of methanol and tetrahydrofuran (THF), optionally wherein the mixture is a 2:1 mixture of methanol and THF; or (iii) in water.

9. The process of any one of claims 5 to 8, wherein in step (a) 20 to 100 moles of trimethylamine are used per mole of compound of Formula (I) or salt thereof, optionally wherein 30 to 80 moles of trimethylamine are used per mole of compound of Formula (I) or salt thereof, optionally wherein 40 to 60 moles of trimethylamine are used per mole of compound of Formula (I) or salt thereof, optionally wherein 50 moles of trimethylamine are used per mole of compound of Formula (I) or salt thereof.

10. The process of any one of claims 5 to 9, wherein step (a) is performed at a temperature of 30° C. to 60° C., optionally 40° C. to 50° C.

11. The process of any one of claims 5 to 10, wherein the reaction of step (a) is allowed to proceed to completion and/or is allowed to proceed for 18 to 30 hours, optionally 22 to 26 hours.

12. The process of any one of claims 5 to 11, wherein step (b) comprises reacting the compound of Formula (II) or a salt thereof with a base, optionally wherein the base is sodium methoxide, potassium carbonate, or lithium hydroxide, preferably sodium methoxide.

13. The process of claim 12, wherein step (b) is performed in a protic polar solvent, optionally which is methanol, ethanol, n-propanol, isopropanol, or a mixture of any of the foregoing, preferably methanol.

14. The process of any one of claims 5 to 13, wherein the compound of Formula (I) or salt thereof is the product of a process comprising reacting a compound of Formula (0a) 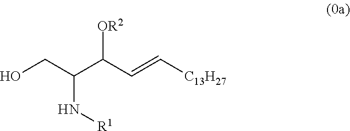
or a salt thereof with

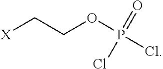

15. The process of any one of claims 5 to 14, wherein the compound of Formula (I) or salt thereof is the product of a process comprising reacting a compound of Formula (0c) 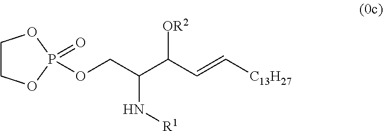
or a salt thereof with MgX₂.

16. The process of any one of claims 5 to 15, further comprising isolating and/or purifying the sphingomyelin or a salt thereof.

17. Sphingomyelin or salt thereof produced by the process of any one of claims 5 to 16, optionally having a purity of at least 99% as measured by high performance thin layer chromatography.

18. A process for synthesizing a protected sphingomyelin or a salt thereof, comprising reacting a compound of Formula (I) 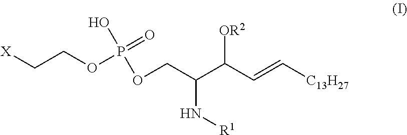
or a salt thereof with trimethylamine, optionally wherein the trimethylamine is liquid, to produce a compound of Formula (II) 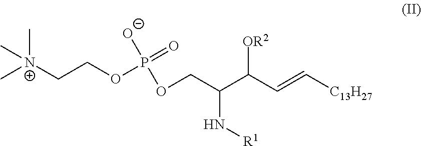
or a salt thereof, wherein
X is halogen;
R¹ is an acyl group having 3 to 36 carbon atoms and zero to six carbon-carbon double bonds; and
R² is a protecting group.

19. The process of claim 18, which comprises reacting a compound of Formula (Ia) 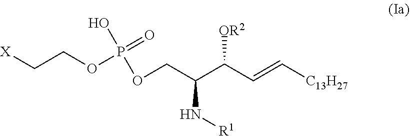
or a salt thereof with trimethylamine, optionally wherein the trimethylamine is liquid, to produce a compound of Formula (IIa) 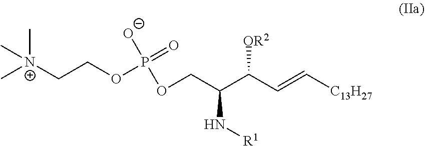
or a salt thereof.

20. The process of claim 18 or claim 19, wherein X is Br; R¹ is palmitoyl, myristoyl, stearoyl, oleoyl, linoleoyl, linolenoyl, arachidonoyl, or eicosapentaenoyl, preferably palimitoyl; and/or R² is a benzoyl group.

21. The process of any one of claims 18 to 20, which comprises reacting the compound of Formula (I) or a salt thereof with trimethylamine in methanol; in a mixture of methanol and tetrahydrofuran (THF); or in water.

22. The process of any one of claims 18 to 21, wherein 20 to 100 moles of trimethylamine are used per mole of compound of Formula (I) or salt thereof, optionally wherein 30 to 80 moles of trimethylamine are used per mole of compound of Formula (I) or salt thereof, optionally wherein 40 to 60 moles of trimethylamine are used per mole of compound of Formula (I) or salt thereof, optionally wherein 50 moles of trimethylamine are used per mole of compound of Formula (I) or salt thereof.

23. The process of any one of claims 18 to 22, wherein the reaction is performed at a temperature of 30° C. to 60° C., optionally 40° C. to 50° C.

24. The process of any one of claims 18 to 23, wherein the reaction is allowed to proceed to completion and/or is allowed to proceed for 18 to 30 hours, optionally 22 to 26 hours.

25. The process of any one of claims 18 to 24, wherein the compound of Formula (I) or salt thereof is the product of a process comprising reacting a compound of Formula (0a) 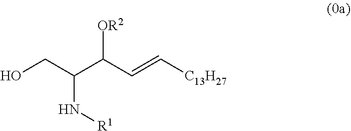
or a salt thereof with

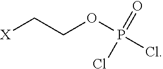

26. The process of any one of claims 18 to 25, wherein the compound of Formula (I) or salt thereof is the product of a process comprising reacting a compound of Formula (0c) 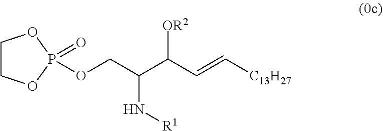
or a salt thereof with MgX₂.

27. A process for making a compound of Formula (I): 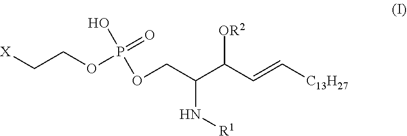
or a salt thereof, wherein:
X is halogen;
R¹ is an acyl group having 3 to 36 carbon atoms and zero to six carbon-carbon double bonds; and
R² is a protecting group,
the process comprising reacting a compound of Formula (0a) 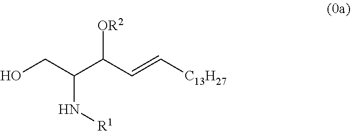
or a salt thereof with

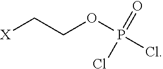

28. The process of claim 27, which comprises reacting a compound of Formula (0b) 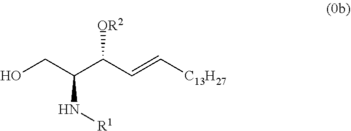
or a salt thereof with

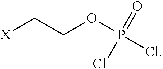

29. The process of claim 27 or claim 28, wherein X is Br; R¹ is palmitoyl, myristoyl, stearoyl, oleoyl, linoleoyl, linolenoyl, arachidonoyl, or eicosapentaenoyl, preferably palmitoyl; and/or R² is a benzoyl group.

30. A process for making a compound of Formula (I): 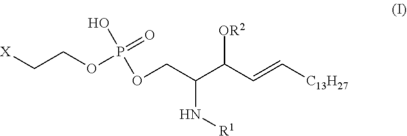
or a salt thereof, wherein:
X is halogen;
R¹ is an acyl group having 3 to 36 carbon atoms and zero to six carbon-carbon double bonds; and
R² is a protecting group,
the process comprising reacting a compound of Formula (0c) 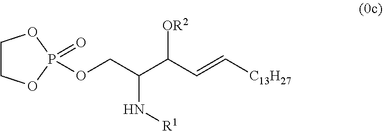
or a salt thereof with MgX₂.

31. The process of claim 30, which comprises reacting a compound of Formula (0d) 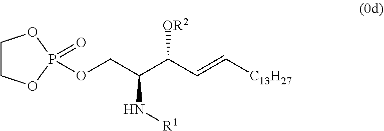
or a salt thereof with MgX₂.

32. The process of claim 30 or claim 31, wherein X is Br; R¹ is palmitoyl, myristoyl, stearoyl, oleoyl, linoleoyl, linolenoyl, arachidonoyl, or eicosapentaenoyl, preferably palmitoyl; and/or R² is a benzoyl group.

33. The process of claim 31 or claim 32, wherein the molar ratio of the compound of Formula (0c) to MgX₂ is 1:1 to 1:3, 2:1 to 1:2, optionally 1:1, 1:1.5, or 1:2.

34. The process of any one of claims 31 to 33, wherein the reaction is performed at a temperature of 20° C. to 40° C., optionally 25 to 35° C.

35. The process of any one of claims 30 to 34, wherein the compound of Formula (0c) is the product of a process comprising reacting 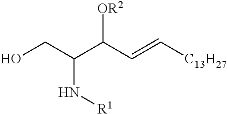
with 2-chloro-2-oxo-1,3,2-dioxaphospholane (CCP), optionally wherein the molar ratio of 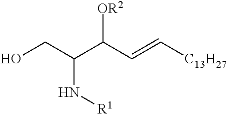
to CCP in the reaction to form the compound of Formula (0c) is 3:1 to 1:3, optionally 2:1 to 1:2, optionally 1:1 or 1:1.5.

36. The process of claim 35, wherein the reaction to form the compound of Formula (0c) is performed at 10° C. to 20° C. or 0 to 10° C.

37. A lipid binding protein-based complex comprising the sphingomyelin or salt thereof of claim 17.

38. The lipid binding protein-based complex of claim 37, which is CER-001.

39. The lipid binding protein-based complex of claim 37 or claim 38, which is a carrier for a drug.

40. A pharmaceutical composition comprising the lipid binding protein-based complex of any one of claims 37 to 39 and a pharmaceutically acceptable excipient.

41. A process for making a lipid binding protein-based complex comprising combining the sphingomyelin or salt thereof of claim 17 with a lipid binding protein.

42. The process of claim 41, which comprises combining the sphingomyelin or salt thereof and the lipid binding protein to form a mixture and, subsequently, thermal cycling the mixture.

43. The process of claim 41 or claim 42, wherein the lipid binding protein is apolipoprotein A-I (ApoA-I), optionally wherein the ApoA-I is recombinant.

44. The process of claim 43, wherein the amino acid sequence of the ApoA-I comprises amino acids 25 to 267 of SEQ ID NO:1.

45. The process of claim 43 to claim 44, wherein the ApoA-I is pre-complexed with DPPG prior to combining with the sphingomyelin or salt thereof, optionally wherein the weight ratio of ApoA-I to lipid is 1:2.7 and/or wherein the sphingomyelin to DPPG weight ratio is 97:3.

46. The process of claim 45, which comprises combining the ApoA-I pre-complexed with DPPG with the sphingomyelin or salt thereof to form an ApoA-I/DPPG/sphingomyelin mixture and thermal cycling the ApoA-I/DPPG/sphingomyelin mixture between 57° C.±10° C. and 37° C.±10° C.

47. A lipid binding protein-based complex obtained or obtainable by the process of any one of claims 41 to 46.
